(19) United States
(12) Patent Application Publication
(10) Pub. No.: US 2024/0269079 A1
(43) Pub. Date: Aug. 15, 2024

(54) MELATONIN FORMULATION IN SOLID DOSAGE FORM

(71) Applicant: AGB-Pharma AB, Lund (SE)

(72) Inventor: Staffan WAXEGÅRD, Lund (SE)

(21) Appl. No.: 18/568,527

(22) PCT Filed: Jun. 9, 2022
(86) PCT No.: PCT/EP2022/065690

§ 371 Date: Dec. 8, 2023

(30) Foreign Application Priority Data
Jun. 9, 2021 (EP) 21178508.4

(51) Int. Cl. A61K 9/20; A61K 31/4045

(52) U.S. Cl. CPC A61K 9/2054; A61K 9/2009; A61K 9/2018; A61K 9/2095; A61K 31/4045

(57) ABSTRACT

The present invention relates to a melatonin formulation in solid dosage form with a rapid-release profile and methods for preparation thereof. In particular, the present invention relates to a melatonin tablet that can be prepared in a dry mixing process without the need for any solvents, such as organic solvents.

### TECHNICAL FIELD OF THE INVENTION

The present invention relates to a melatonin formulation in solid dosage form with a rapid-release profile and methods for preparation thereof. In particular, the present invention relates to a melatonin tablet that can be prepared in a dry mixing process without the need for any solvents, such as organic solvents.

### BACKGROUND OF THE INVENTION

Sleep disorders affect hundreds of millions of people worldwide on a continuous basis, many of which suffers from negative impact on quality of life, lack of productivity and high health care utilization. The most prevalent sleep disorder is insomnia or more literally the inability to sleep. Insomnia has various etiologies and degrees of severeness and is estimated to affect up to 6-12% of the adult population and as much as between 15-25% of children. Accordingly, there is a massive need for providing treatment of sleep disorders.

Traditionally, synthetic hypnotics such as barbiturate and later benzodiazepines and quinazolinones have been used to induce sleep. Predominantly, benzodiazepines enhancing the effect of the neurotransmitter gamma-aminobutyric acid (GABA) at the GABA_{A} receptor have been prescribed to individuals suffering from a sleep disorder. However, long-term use of benzodiazepines is not recommended due to the risk of dependence, daytime sedation and respiratory depression, and at best only intermittent administration at the lowest effective dose is commended. Benzodiazepines also generally worsen sleep quality by increasing light sleep and decreasing deep sleep.

In response to the safety concerns revolving benzodiazepines, non-benzodiazepine hypnotics (or so-called Z-drugs) were introduced in the early 1990's. These drugs are “benzodiazepine-like” in nature and display similar pharmacodynamic profiles. Although the use of non-benzodiazepines has become more popular, the alleged improved efficacy is still debated and side-effects similar to those of benzodiazepines are frequent.

In contrast to the synthetic hypnotics, melatonin is a naturally occurring indole hormone released by the pineal gland located in the brain. It is released primarily at night and it is well-established that melatonin is involved in modulation of the circadian rhythm regulating the sleep-wake cycle, asserting its effect predominantly through its interaction with the melatonin receptors. Melatonin has also been found to modulate sleep patterns related to seasonal cycles. Additionally, melatonin is also involved in other biological and physiological regulation of body functions, some of which are related to its role as an antioxidant and free radical scavenger.

Melatonin is a neurohormone with the chemical identity N-acetyl-5-methoxytryptamine and is made from tryptophan via serotonin as an intermediate. In the past, melatonin was derived from bovine pineal tissue, but today it is mainly synthetic, which limits the risk of contamination or the means of transmitting infectious material.

Use of melatonin to induce sleep has several advantages over conventional synthetic hypnotics. First, melatonin is naturally metabolized with blood levels returning to normal daytime levels within 6-8 hours of administration. Therefore, melatonin does not cause adverse effects the day following administration as is in many instances an issue with conventional hypnotics. Importantly, melatonin does not present the same risk of dependency as conventional hypnotics and being a naturally occurring and endogenous compound does not induce amnesia effects as benzodiazepines.

Given the many benefits of melatonin as a sleep-inducing agent its use as pharmaceutical has been widely explored. Melatonin may be provided in either solid dosage forms or in liquid dosage forms. The latter has the drawback that liquid formulations have shorter shelf-life than solid dosage forms and therefore are not very convenient for the consumer. Solid dosage forms on the other hand should be provided as rapid release formulations that cause blood levels of melatonin to reach their peak in about an hour, thereby facilitating a quick sleep-inducing effect that is comparable to liquid formulations.

Unfortunately, the manufacture of rapid release formulations of melatonin has proved to be challenging. Specifically, intrinsic properties such as the high degree of aggregation and adhesiveness of melatonin makes formulation of solid dosage forms with the required homogeneity a difficult task. Especially, it is difficult to obtain sufficient homogeneity in dry blends within mixing periods that are short enough to be industrially feasible. The problem with insufficient homogeneity may be addressed by dissolving and dispersing melatonin in a solvent as is done in wet granulation. However, this approach poses two challenges. Firstly, melatonin has low solubility in aqueous solutions, which likely necessitates the use of an organic solvent, such as acetone or ethanol. However, organic solvents are not appreciated in industrial production of pharmaceutical formulations due to safety hazards. Secondly, the addition of a wet processing step, such as wet granulation, significantly reduce large scale production capacity of a melatonin formulation in solid dosage form.

Hence, it would be advantageous to provide a method for preparing a rapid-release solid dosage form of homogenously distributed melatonin. Specifically, such a method may advantageously comprise no wet process steps and thereby enable enhanced large scale manufacture capacity. Providing a fast method that is industrially attractive would be particularly advantageous.

### SUMMARY OF THE INVENTION

Formulations for mitigating sleep disorders is of great importance when put in perspective of how many lives are affected every single day. The naturally occurring neurohormone melatonin may be used to induce sleep without the severe risk of adverse effects associated with conventional synthetic hypnotics. However, the provision of melatonin as solid dosage forms with swift effect has been difficult and especially scaling of its production is challenging. Thus, there is an unmet need for processes that allow large scale manufacture of melatonin formulated in high-quality solid dosage forms with a rapid-release profile.

The present invention relates to a method for manufacture of a rapid release melatonin formulation in solid dosage form. The method comprises a series of dry mixing steps that obviates any cumbersome wet processing steps and therefore is particular suitable for large scale production. Dry processing of the formulation is designed to achieve a homogeneous distribution of ingredients throughout a batch of solid dosage forms, while at the same time delivering solid dosage forms according to required standards. The present invention also provides melatonin formulations in solid dosage form that provide a rapid effect to combat sleep disorders.

Thus, an object of the present invention relates to the provision of a method for preparing solid dosage forms with accurately defined doses of melatonin that rapidly releases and assert its effect upon ingestion by an individual.

In particular, it is an object of the present invention to provide a method for large scale production of solid dosage forms comprising melatonin that exclusively involves dry processing of the ingredients.

Thus, an aspect of the present invention relates to a method for manufacture of a rapid release melatonin formulation in solid dosage form, said method comprising:
i) a first adding step comprising adding first components into a mixing container to form a first mixture, wherein the first components comprise one or more fillers and optionally one or more glidants and/or disintegrants,
ii) a stepwise adding step comprising providing a first amount of microcrystalline cellulose and a total amount of micronized melatonin, and adding said first amount of microcrystalline cellulose and said total amount of micronized melatonin into the mixing container stepwise in multiple portions,
iii) a second adding step comprising adding second components into the mixing container to form a second mixture, wherein the second components comprise a second amount of microcrystalline cellulose and optionally one or more fillers, glidants and/or disintegrants, and wherein the second mixture comprises micronized melatonin, microcrystalline cellulose, filler, glidant and disintegrant
iv) a third adding step comprising adding magnesium stearate into the mixing container to form a final mixture, and
v) a processing step comprising processing the final mixture to a solid dosage form,
   wherein at least one of the first and/or second adding step comprises adding one or more glidants, and
   wherein at least one of the first and/or second adding step comprises adding one or more disintegrants.

Another aspect of the present invention relates to a rapid release melatonin formulation obtainable by a method as described herein.

Yet another aspect of the present invention relates to a rapid release melatonin formulation comprising:
0.2-3 wt % micronized melatonin,
20-35 wt % microcrystalline cellulose,
65-75 wt % of one or more fillers,
0.2-0.8 wt % of one or more glidants,
1-3 wt % of one or more disintegrants, and
at least 3 wt % magnesium stearate,
   wherein the formulation is provided as solid dosage form and wt % is with respect to the total weight of the formulation.

Still another aspect of the present invention relates to a rapid release melatonin formulation as described herein for use as a medicament.

A further aspect of the present invention relates to a rapid release melatonin formulation as described herein for use in the treatment of sleep disorders.

A still further aspect of the present invention relates to a kit of parts comprising:
a plurality of solid dosage forms comprising a rapid release melatonin formulation as described herein, and
optionally, instructions for use.

### BRIEF DESCRIPTION OF THE FIGURES

FIG. 1 shows dissolution profiles for N4, N7 and N10, tablets compressed at different compression forces; x-axis: time, min; y-axis: percentage dissolved API. N4; 4.5, 8 and 10 kN; N7: 5.3 and 9 kN; N10: 5 and 8 kN.

FIG. 2 shows dissolution profiles of melatonin formulations with or without disintegrant in the formulation. Displayed is N10 at compression forces of 5 kN (3 mg-5 kN-0% Cam) and 8 kN (3 mg-8 kN-0% Cam), respectively, as well as exp. no. 1 (3 mg-0% Cam) compressed at 5 kN and 8 kN, respectively.

The present invention will in the following be described in more detail.

### DETAILED DESCRIPTION OF THE INVENTION

### Definitions

Prior to outlining the present invention in more details, a set of terms and conventions is first defined:

### Melatonin

In the present context, the term “melatonin” refers to the neurohormone released by the pineal gland in the brain. Melatonin has the chemical identity N-acetyl-5-methoxytrypamine. One form of melatonin is micronized melatonin, wherein the melatonin particles have been comminuted to decrease particle size.

### Micronized Melatonin

In the present context, the term “micronized melatonin” refers to a population of melatonin particles with a D90 of 30 μm or less when measured using laser diffraction. D90 describes the value at which 90% of the particles within the population have a diameter below this value.

Particle size and particle size distribution (PSD) can be determined using a Malvern Mastersizer 2000 from Malvern Instruments with a measuring cell Hydro 2000 μP. This particle size analyzer is based on laser diffraction and can accurately determine the PSD of solid particulate melatonin. Low angle laser light scattering is responsive to the volume of a particle and yields a volume-average particle size, which is equivalent to the weight-average particle size as the density is held constant.

Settings of the Malvern Mastersizer 2000 (Hydro 2000S measuring cell) were as follows.
Analysis model: General purpose (Particle shape=irregular)
Optical properties material: RI=1.60 absorption=0
Pump speed: 2500 rpm
Ultrasonics: None
Dispersant name: Heptane, heptane RI: 1.39
Melatonine F RI: 1.6 Absorption=0
Sample quantity: 50 mg
Obscuration: 5-25%
Measurements time: 60 seconds
Background time: 10 seconds
Aliquots: 1 for SOP
Measurements: 3 for aliquot
Delay: 10 seconds
Create average results: yes
Clean mode: Manual full wash

### Microcrystalline Cellulose

In the present context, the term “microcrystalline cellulose” or MCC″ refers to isolated crystalline regions of microfibrils of naturally occurring polymers composed of glucose units connected by 1-4 beta glycosidic bonds. Thus, MCC has a fibrous structure and functions as a binder.

Binders hold the ingredients in a solid dosage form together and ensure that the solid dosage forms can be formed with the required mechanical strength.

### Filler

In the present context, the term “filler” refers to a substance that provides volume to a solid dosage form with a low active dose. Thus, fillers are added to make handling of small amounts of active ingredients more convenient. Fillers are typically inactive.

### Glidant

In the present context, the term “glidant” refers to a substance that is added to a solid dosage form to improve flowability properties. Generally, glidants promote powder flow by reducing interparticle friction and cohesion.

### Disintegrant

In the present context, the term “disintegrant” refers to a substance which causes the solid dosage form to disintegrate upon exposure to a changed environment, such as a solvent. Generally, disintegrants expand and dissolve when the are exposed to a solvent, e.g. in the digestive tract, thereby releasing the active ingredient for absorption and promoting bioavailability.

### Lubricant

In the present context, the term “lubricant” refers to a substance which counteracts that the final powder mixture adheres to the surface of the equipment during final processing into solid dosage forms. Thus, lubricants can prevent ingredients from sticking to e.g. tablet punches and ensure that compression and ejection of tablets can occur with low friction between the solid and the die wall. Lubricants may also contribute to preventing ingredients from clumping together.

### Rapid Release Formulation

In the present context, the term “rapid release formulation” refers to a formulation designed to release the active ingredient shortly after ingestion by an individual. This type of formulation promotes quick bioavailability of the active ingredient with few or no rate controlling features, such as special coatings or the like.

With the specific regard to tablets, the term “immediate release tablet” is often used. Accordingly, the terms “rapid release formulation” and “immediate release formulation” are used interchangeably herein.

### Solid Dosage Form

In the present context, the term “solid dosage form” refers to the physical form of the melatonin formulation. Thus, the solid dosage form may include, but are not limited to, tablets, pills, lozenges, capsules, and pastilles.

The solid dosage forms can be provided as unit doses, which corresponds to the products in the form in which they are marketed, with a specific mixture of active ingredients and other ingredient (e.g. filler, binder, glidant, lubricant, disintegrant), in a particular configuration (such as a tablet for example), and apportioned into a particular dose.

### Flavouring Agent

In the present context, the term “flavouring agent” refers to a substance that is used to disguise any unpleasant taste caused by an active ingredient or other ingredients and improve the organoleptic experience of the consumer. Flavouring agents may be of natural or artificial origin.

### Weight Percentage (wt %)

In the present context, the term “weight percentage” or “wt %” refers to the relative weight of the respective ingredient (melatonin, filler, binder, glidant, lubricant, disintegrant) with respect to the total weight of the formulation or solid dosage form as specified, unless otherwise defined.

Thus, when explicitly specified, the weight percentage may be defined with respect to separate ingredients of the formulation.

### Homogeneity

In the present context, the term “homogeneity” refers to the degree of uniformity with which the active ingredient is distributed across the formulation and in the final solid dosage forms. Thus, a high degree of homogeneity means that close to equal amounts of active ingredient is present in each solid dosage form.

Homogeneity may be assessed by quantifying the “uniformity of dosage units”. This is done in accordance with European Pharmacopoeia 10^{th} Edition, 2.9.40 Uniformity of dosage units.

Briefly, homogeneity is evaluated by determining relative standard deviation (RSD, %) of 10 tablets. Assay is carried out by reversed phase liquid chromatography and UV detection at 285 nm.

The following equipment were used for determination of homogeneity.
Analytical column: Waters Acquity HSS T3 C18 2.1×100 mm, 1.8 μm
Pump: Waters Acquity ARC Quaternary Solvent Manager (QSM)
Autosampler: Waters Acquity ARC Sample Manager (SM)
Column oven: Waters Acquity ARC Column Manager (CM)
Detector: Waters Acquity ARC PDA Detector
Software: Empower
Syringe filter: RC Membrane filter, 0.2 μm, 26 mm syringe filters, non-sterile, PP-housing, Luer/slip, Phenomenex

The terms “homogeneity” and “uniformity” are used interchangeably herein.

### Disintegration Time

In the present context, the term “disintegration time” refers to the time the solid dosage form takes to disintegrate in a specified test in accordance with European Pharmacopoeia 10^{th} Edition, 2.9.1 Disintegration of Tablets and Capsules (Ph.Eur.2.9.1).

The disintegration time of the tablets herein is measured with a Erweka ZT53 disintegration tester.

In brief, tablets are placed in a basket at the highest position and then the timer and equipment are started. When the tablet has passed through the mesh at the bottom of the basket the time is noted. The medium used was de-ionized water at 37° C.

### Friability

In the present context, the term “friability” refers to the tendency of the solid dosage form to break into smaller fractions under duress. The friability can be determined in accordance with European Pharmacopoeia 10^{th} Edition, 2.9.7 Friability of uncoated tablets (Ph.Eur.2.9.7). The test is intended to determine, under defined conditions, the friability of uncoated tablets, the phenomenon whereby tablet surfaces are damaged and/or show evidence of lamination or breakage when subjected to mechanical shock or attrition.

The friability of the tablet herein is measured with a Erweka TA20 Laboratory Friability Tester.

Briefly, friability testing is performed in rotating testing drums, designed according to the pharmacopeia. The measured parameter is weight loss before and after testing and tumbling the tablets at a particular time and speed. A number of tablets are randomly selected, weighed together and transferred to the friability tester. The drums turn at a consistent 25 rpm. As the product in the drum tumbles against the drum's internal vane, its loss due to breakage or chipping can be measured. The test run for 4 minutes and then the tablets are de-dusted and weighed again. The amount lost is then calculated as a percentage.

### Resistance to Crushing

In the present context, the term “resistance to crushing” refers to the ability of the solid dosage form to withstand a force applied thereupon. Resistance to crushing is defined by the force needed to disrupt the solid dosage form and can be measured according to European Pharmacopoeia 10^{th} Edition, 2.9.8 Resistance to crushing of tablet (Ph. Eur.2.9.8).

Resistance to crushing may be given as Newton or kilopond (kP). The relation between those is 1 kP=9.81 N.

Resistance to crushing of the tablet herein is measured as an average of 10 tablets. All tablets are placed with the embossed side up.

“Resistance to crushing” is also called “breaking force” and the two terms are used interchangeably herein.

### Tensile Strength

In the present context, the term “tensile strength” refers to the maximum stress that the solid dosage form can sustain while being stretched or pulled before the solid dosage form break apart. The tensile strength can be calculated from the breaking force (also previously termed “hardness”) and tablet dimensions, allowing comparison of the mechanical strength of different sizes of solid dosage forms.

Thus, the tensile strength of a cylindrical tablet may be calculated by the equation: σₓ=(2*F/(π*D*H)), wherein ox is the tensile strength, F is the breaking force, D is the tablet diameter, and H is the tablet thickness.

The breaking force of the tablets herein is measured with a Pharmatest 302 tablet hardness tester. Breaking force is tested to assure that the tablet's strength will survive all further processes, such as dedusting, coating and packaging.

### Blending

In the present context, the term “blending” refers to the act of mixing two or more ingredient with each other. Blending can be performed in a suitable mixing container. Blending may be accomplished by shear mixing under agitation or stirring, which can be facilitated by a rotor or impeller.

The terms “blending” and “mixing” are used interchangeably herein.

### Sieving

In the present context, the term “sieving” refers to a process for separating particles of different sizes. Sieving can be conducted by passing a powder through a screen with a defined mesh size. Coarse particles that are larger than the mesh size are separated or broken up by grinding against one another and the mesh openings.

### Direct Compression

In the present context, the term “direct compression” refers to a process wherein tablets are directly compressed from a powder mixture of an active ingredient and other ingredients (e.g. filler, binder, glidant, lubricant, disintegrant). Accordingly, direct compression does not require any pre-treatment of the powder mixture, such as dry or wet granulation process steps.

### About

Wherever the term “about” is employed herein in the context of amounts, for example absolute amounts, such as numbers, purities, weights, sizes, etc., or relative amounts (e.g. percentages, equivalents or ratios), timeframes, and parameters such as temperatures, pressure, etc., it will be appreciated that such variables are approximate and as such may vary by ±10%, for example±5% and preferably ±2% (e.g. ±1%) from the actual numbers specified. This is the case even if such numbers are presented as percentages in the first place (for example ‘about 10%’ may mean±10% about the number 10, which is anything between 9% and 11%).

### Melatonin Formulation in Solid Dosage Form and Method for the Production Thereof

As outlined herein, sleep disorders affect millions of individuals worldwide every single day. Synthetic means for mitigating the negative impact of sleep disorders have existed for decades but in many cases are provided as liquid solutions that are impractical to handle and/or are associated with severe adverse effects.

Melatonin is a naturally occurring neurohormone that is an alternative active ingredient to combat sleep disorders that does not cause the severe adverse effects associated with the synthetic counterparts. Unfortunately, it has proven difficult to produce rapid release solid dosage forms of melatonin on a large scale, mainly due to the high degree of aggregation and adhesiveness of melatonin. Dry mixing in general tends to be complicated by the inherent cohesiveness and resistance to movement between the individual particles and substantial segregation due to differences in size, shape, and density of the dry particles. These general challenges with dry mixing and the intrinsic properties of melatonin cause uneven distribution of content across a batch of solid dosage forms. While inhomogeneity of contents may be handled by dissolving or dispersing melatonin in a solvent, such wet processing step in very undesirable from a large-scale manufacturing point of view. Notably, dry processing in which solid dosage forms are directly compressed from a powder blend without the need for solvents is advantageous as it requires less machinery, reduced number of personnel, fewer unit operations and significantly less processing time.

Herein is provided a method for producing solid dosage forms of melatonin with uniformly distributed content that relies exclusively on dry processing steps, including the stepwise addition of micronized melatonin together with microcrystalline cellulose (MCC). The method produces high quality solid dosage forms, e.g. tablets, with fast disintegration which are easy to store and handle.

Thus, an aspect of the present invention relates to a method for manufacture of a rapid release melatonin formulation in solid dosage form, said method comprising:
i) a first adding step comprising adding first components into a mixing container to form a first mixture, wherein the first components comprise one or more fillers and optionally one or more glidants and/or disintegrants,
ii) a stepwise adding step comprising providing a first amount of microcrystalline cellulose and a total amount of micronized melatonin, and adding said first amount of microcrystalline cellulose and said total amount of micronized melatonin into the mixing container stepwise in multiple portions,
iii) a second adding step comprising adding second components into the mixing container to form a second mixture,
   wherein the second components comprise a second amount of microcrystalline cellulose and optionally one or more fillers, glidants and/or disintegrants, and wherein the second mixture comprises micronized melatonin, microcrystalline cellulose, filler, glidant and disintegrant
iv) a third adding step comprising adding magnesium stearate into the mixing container to form a final mixture, and
v) a processing step comprising processing the final mixture to a solid dosage form,
   wherein at least one of the first and/or second adding step comprises adding one or more glidants, and
   wherein at least one of the first and/or second adding step comprises adding one or more disintegrants.

The method provided herein is based on the use of melatonin in micronized form to ensure high quality of the solid dosage forms. Melatonin in micronized form differs from traditional melatonin in that it is processed to particles of smaller size. This processing impacts particle characteristics such as shape, size and size distribution. Inadvertent variability in these characteristics may cause issues with weight, content uniformity, segregation, compression, and dissolution of the final solid dosage forms. Presence of a minority of excessively large particles may for instance cause unintentional low weight of the solid dosage forms because the dies are filled volumetrically or extend the dissolution profile due to a diminished surface area of the solid dosage form.

Micronized melatonin has a narrower size distribution than traditional melatonin, which at times can have a multimodal size distribution exacerbating the unwanted effects described above. Utilizing micronized melatonin therefore assists in ensuring the required uniform distribution of content in the powder blend and ultimately in the solid dosage forms. Moreover, the small particle size of micronized melatonin improves absorptivity of the active ingredient (melatonin) upon release from the solid dosage form.

Therefore, an embodiment of the present invention relates to the method as described herein, wherein the micronized melatonin has a D90 value of 30 μm or less, preferably 20 μm or less, more preferably 10 μm or less.

To facilitate efficient distribution of melatonin throughout the powder blend and prevent aggregation of melatonin, the micronized melatonin is premixed with microcrystalline cellulose (MCC) before addition to the mixing container. MCC has a fibrous structure to which melatonin adheres readily as compared to more spherical components, such as mannitol. By premixing the micronized melatonin with MCC prior to contact with a filler, therefore enhance homogeneous distribution of melatonin in the powder blend.

It has been found to be favorable to premix the micronized melatonin only with a fraction of the MCC in the formulation and further adjust the ratio of MCC to filler in order to improve uniformity of the powder blend.

Thus, an embodiment of the present invention relates to the method as described herein, wherein the ratio between said first amount of microcrystalline cellulose and said one or more fillers of the first components is in the range of 1:8 to 1:12 wt %/wt %, preferably approximately 1:10 wt %/wt %, with respect to the total weight of the solid dosage form.

Another embodiment of the present invention relates to the method as described herein, wherein the ratio between said first amount of microcrystalline cellulose and said second amount of microcrystalline cellulose is in the range of 1:4 to 1:10 wt %/wt %, preferably approximately 1:5 wt %/wt %, with respect to the total weight of microcrystalline cellulose in the solid dosage form.

Premixing of micronized melatonin and MCC can be achieved by stepwise addition of portions comprising both micronized melatonin and MCC. The amount of micronized melatonin and MCC in each portion may be varied from e.g. three to eight portions, but higher number of portion could also be feasible although inefficient from a practical point of view. Typically, the fractions of micronized melatonin and MCC in a portion are of equal proportion with respect to the first amount of MCC and total amount of micronized melatonin, respectively, added during the stepwise adding step. In practice, this means that a portion may comprise e.g. 20 wt % of the first amount of MCC and 20 wt % of the total amount of micronized melatonin. Each portion of the multiple portions is not limited to comprising the same amount of MCC and micronized melatonin. If, by example, three portions are added in the stepwise adding step, the portions may comprise e.g. 25 wt %, 50 wt % and 25 wt %, of the first amount of MCC and/or total amount of micronized melatonin, respectively. However, in a variant of the method, each portion comprises the same amount of MCC and micronized melatonin. In this case, e.g. five portions would each comprise 20 wt % of the first amount of MCC and/or total amount of micronized melatonin.

Thus, an embodiment of the present invention relates to the method as described herein, wherein each portion of the stepwise adding step comprises a fraction of said first amount of microcrystalline cellulose and a fraction of said total amount of micronized melatonin.

Another embodiment of the present invention relates to the method as described herein, wherein each portion of the stepwise adding step comprises between 10-50 wt % of said first amount of microcrystalline cellulose, such as 15-40 wt % of said first amount of microcrystalline cellulose, such as 15-25 wt % of said first amount of microcrystalline cellulose, preferably approximately 20 wt % of said first amount of microcrystalline cellulose.

A further embodiment of the present invention relates to the method as described herein, wherein each portion of the stepwise adding step comprises between 10-50 wt % of said total amount of micronized melatonin, such as 15-40 wt % of said total amount of micronized melatonin, such as 15-25 wt % of said total amount of micronized melatonin, preferably approximately 20 wt % of said total amount of micronized melatonin.

Yet another embodiment of the present invention relates to the method as described herein, wherein the fractions of micronized melatonin and MCC in a portion are of equal proportion with respect to the first amount of MCC and total amount of micronized melatonin, respectively.

Still another embodiment of the present invention relates to the method as described herein, wherein said multiple portions are three portions, four portions, five portions, six portions, seven portions, or eight portions, preferably five portions.

Sieving of the ingredients into the mixing container can further induce homogenous distribution of content. As part of the premixing of micronized melatonin, a pre-mixing screen may be used when introducing micronized melatonin and MCC into the mixing container. The mesh size of the pre-mixing screen can be adjusted to the process. In general, a smaller screen results in a better distribution of content, but also comes with longer processing time.

Therefore, an embodiment of the present invention relates to the method as described herein, wherein the stepwise adding step comprises sieving the total amount of micronized melatonin and the first amount of microcrystalline cellulose through a pre-mixing screen.

Another embodiment of the present invention relates to the method as described herein, wherein the pre-mixing screen has a mesh size in the range of 0.8-1.2 mm, preferably approximately 1 mm.

Premixing of micronized melatonin and MCC is enhanced when the two ingredients are sieved through the pre-mixing screen together. Thus, for each portion to be added, an amount of MCC and an amount of micronized melatonin may be charged to the pre-mixing screen and simultaneously passed through and into the mixing container by agitation of the pre-mixing screen.

Accordingly, an embodiment of the present invention relates to the method as described herein, wherein each portion of the stepwise adding step comprises a fraction of said first amount of microcrystalline cellulose and a fraction of said total amount of micronized melatonin that are sieved together through the pre-mixing screen.

In a variant of the method, the micronized melatonin and MCC is mixed in a pre-mix blend before addition to the pre-mixing screen. In this variant of the method, micronized melatonin and MCC does not have to be charged onto the pre-mixing screen separately but is charged as a pre-mix blend. This can save some processing time.

Thus, an embodiment of the present invention relates to the method as described herein, wherein the stepwise adding step comprises providing the first amount of microcrystalline cellulose and the total amount of micronized melatonin as a pre-mix blend.

Another embodiment of the present invention relates to the method as described herein, wherein each portion of the stepwise adding step comprises between 10-35 wt % of said pre-mix blend, such as 15-25 wt % of said pre-mix blend, preferably approximately 20 wt % of said pre-mix blend.

Besides the active ingredient (melatonin) and MCC, the solid dosage forms comprise other ingredients, such as filler(s), glidant(s), and disintegrant(s), that are typically included in solid dosage forms. Many specific ingredients may be used, and the method presented herein is not limited to producing a solid dosage form with any specific filler(s), glidant(s), and disintegrant(s). Moreover, it is contemplated that the solid dosage form in some variants may comprise one or more ingredients aiming at fulfilling the same function.

Fillers are substances that provide volume to the solid dosage form. This is especially important for formulations of low active dose, wherein handling and homogeneous distribution of the active ingredient is challenging. Fillers are also sometimes called diluents.

An embodiment of the present invention relates to the method as described herein, wherein the one or more fillers are selected from the group consisting of mannitol, sorbitol, xylitol, dextrose, sucrose, lactose, polyols, calcium carbonate, calcium sulphate, magnesium oxide, magnesium carbonate, maltodextrin, polymethacrylates, potassium chloride, kaolin, dicalcium phosphate dihydrate, starches, and combinations thereof, preferably mannitol.

Mannitol is a sugar alcohol that is well-tolerated and has favourable characteristics as a filler. Thus, an embodiment of the present invention relates to the method as described herein, wherein the one or more fillers comprise mannitol. A further embodiment of the present invention relates to the method as described herein, wherein mannitol is the only filler.

In variants of the method, the one or more fillers may be added in first and second amounts in different adding steps. For instance, a first amount of a certain filler may be added in the first adding step and the remaining amount of the same filler may be added in the second adding step. Such multiple addition with the same filler may e.g. be when the filler is mannitol. Thus, an embodiment of the present invention relates to the method as described herein, wherein a first amount of filler is added in the first adding step and the remaining amount of the filler is added in the second adding step.

For some applications it can be preferable to abstain from using certain fillers. One such example is lactose, which may cause adverse effects to lactose intolerant individuals. Therefore, an embodiment of the present invention relates to the method as described herein, wherein the solid dosage form does not comprise lactose.

Glidants are important ingredients that promote flowability of the non-compacted powder and improve accuracy of dosing in the solid dosage forms. Any traditional glidant can be used with the method described herein.

Therefore, an embodiment of the present invention relates to the method as described herein, wherein the one or more glidants are selected from the group consisting of colloidal anhydrous silica, sodium stearyl fumarate, magnesium trisilicate, talc, tribasic calcium phosphate and combinations thereof, preferably colloidal anhydrous silica.

Colloidal anhydrous silica is a light, fine, white, amorphous powder that fulfil all regulatory requirements for a glidant. It comprises fine particles of approximately 15 nm. Colloidal anhydrous silica is also known as colloidal silicon dioxide. In addition to improving flow properties, colloidal anhydrous silica has anti-caking properties and improves both hardness and friability of the solid dosage form. Thus, an embodiment of the present invention relates to the method as described herein, wherein the one or more fillers comprise colloidal anhydrous silica. Another embodiment of the present invention relates to the method as described herein, wherein colloidal anhydrous silica is the only glidant.

The ability to rapidly release the active ingredient is important in any solid dosage forms wherein a fast effect is required following ingestion. This is surely the case for solid dosage forms intended to aid the sleep process of an individual. Disintegrants function upon contact with a solvent, such as water, by expanding, swelling, hydrating and/or dissolving to produce a disruptive force in the solid dosage form that ruptures the solid structure thereby releasing the active ingredient for absorption.

Thus, an embodiment of the present invention relates to the method as described herein, wherein the one or more disintegrants are selected from the group consisting of croscarmellose sodium, crospovidone, alginic acid, carboxymethylcellulose calcium, carboxymethylcellulose sodium, sodium starch glycolate, carboxymethyl starch, guar gum, magnesium aluminium silicate, methyl cellulose, polacrilin potassium, pregelatinized starch, sodium alginate and combinations thereof, preferably croscarmellose sodium.

Croscarmellose sodium is an internally cross-linked sodium carboxymethylcellulose. Cross-linking reduces water solubility but allows the material to swell and absorb large amounts of water. Croscarmellose sodium swells 4-8 fold in less than 10 seconds and promotes fast disintegration of the solid dosage form. Thus, bioavailability of the active ingredient is increased as contact with the environment is enhanced. Thus, an embodiment of the present invention relates to the method as described herein, wherein the one or more disintegrants comprise croscarmellose sodium. Another embodiment of the present invention relates to the method as described herein, wherein croscarmellose sodium is the only disintegrant.

The melatonin formulation may be formulated at different strengths of active ingredient. Typical solid dosage forms of melatonin, such as tablets, are formulated in strengths of at least 2 mg, or in some cases 1 mg melatonin. For a solid dosage form of 200 mg, a 1 mg dose of active ingredient corresponds to 0.5 wt % of the total weight of the solid dosage form. The method described herein enables large scale production of rapid release solid dosage forms, such as tablets, with a content of melatonin of less than 1 mg. It is therefore possible to produce solid dosage forms of a wide range of dose strengths using the method described herein. Solid dosage forms of low melatonin strength may be advantageous for specific groups of individuals, such as children wherein less melatonin is needed to reach sufficient blood levels of melatonin to facilitate a quick sleep-inducing effect.

Thus, an embodiment of the present invention relates to the method as described herein, wherein the final mixture comprises 0.2-3 wt % micronized melatonin with respect to the total weight of the solid dosage form.

Another embodiment of the present invention relates to the method as described herein, wherein the final mixture comprises at most 1 wt % micronized melatonin with respect to the total weight of the solid dosage form, such as at most 0.5 wt % micronized melatonin with respect to the total weight of the solid dosage form, preferably at most 0.25 wt % micronized melatonin with respect to the total weight of the solid dosage form.

A further embodiment of the present invention relates to the method as described herein, wherein the final mixture comprises approximately 0.5 wt % micronized melatonin with respect to the total weight of the solid dosage form. Still another embodiment of the present invention relates to the method as described herein, wherein the final mixture comprises approximately 0.25 wt % micronized melatonin with respect to the total weight of the solid dosage form

The remaining ingredients of the solid dosage form is generally inactive, i.e. they do not contribute significantly to the sleep-inducing effect. These ingredients may be included in melatonin formulation in amounts that enhance properties such as disintegration time, friability, resistance to crushing and tensile strength of the solid dosage form.

Therefore, an embodiment of the present invention relates to the method as described herein, wherein the final mixture comprises 20-35 wt % microcrystalline cellulose with respect to the total weight of the solid dosage form.

Another embodiment of the present invention relates to the method as described herein, wherein the final mixture comprises 0.2-0.8 wt % of one or more glidants with respect to the total weight of the solid dosage form.

Yet another embodiment of the present invention relates to the method as described herein, wherein the final mixture comprises 1-3 wt % of one or more disintegrants with respect to the total weight of the solid dosage form.

A still further embodiment of the present invention relates to the method as described herein, wherein the final mixture comprises 65-75 wt % of one or more fillers with respect to the total weight of the solid dosage form.

In variants of the method, the majority of the one or more fillers is added in the first adding step and therefore form part of the first mixture into which the micronized melatonin and MCC is added. The prevalence of filler facilitates good distribution of content. Thus, an embodiment of the present invention relates to the method as described herein, wherein the first mixture comprises at least 80 wt % of the one or more fillers, such as at least 85 wt %, such as at least 90 wt %, such as at least 95 wt %, with respect to the total weight of the one or more fillers in the solid dosage form.

Another embodiment of the present invention relates to the method as described herein, wherein the first mixture comprises all of the one or more fillers comprised in the final mixture.

For some melatonin formulations it may be preferable to add one or more binders to the formulation. Binders help to contain melatonin and other ingredients together after compression. Binders may be added in the first and/or second adding step, preferably the second adding step. The method is not limited to any specific binder. Thus, an embodiment of the present invention relates to the method as described herein wherein the first and/or second components comprises one or more binders.

Another embodiment of the present invention relates to the method as described herein, wherein the one or more binders are selected from the group consisting of acacia, carbomer, dextrin, ethyl cellulose, gelatin, hydroxyethyl cellulose, hydroxypropyl cellulose, liquid glucose, and povidone.

Subsequent to the second adding step the second mixture may be blended within a mixing container. The methods disclosed herein are advantageous in that the manufacturing time can be kept short due to the design of the blending scheme. Thus, the stepwise addition of microcrystalline cellulose and micronized melatonin improves content uniformity. The conditions of the first blending can be adjusted to achieve sufficient mixing of the ingredients and keep the processing time to a minimum. The processing conditions may suitably be adjusted to large scale manufacture conditions.

Accordingly, an embodiment of the present invention relates to the method as described herein, wherein the second adding step is followed by a first blending of the second mixture.

Another embodiment of the present invention relates to the method as described herein, wherein the first blending is performed for a duration of 40-240 seconds, such as 60-180 seconds, such as 100-140 seconds, preferably for approximately 120 seconds.

A further embodiment of the present invention relates to the method as described herein, wherein the first blending is performed at less than 120 rpm, such as at less than 100 rpm, such as at less than 80 rpm, preferably at less than 60 rpm.

Lubricants are added to powder formulations to aid manufacturability of the final solid dosage form. In particular, lubricants ensures that ingredients do not stick to the surface of the equipment, such as punch and die, during processing potentially halting production and compromising the quality of the solid dosage forms due to pitting or other exterior anomalies. For the method described herein, it has been found to be favourable to use magnesium stearate. Thus, an embodiment of the present invention relates to the method as described herein, wherein the final mixture comprises 0.5-2.0 wt % magnesium stearate with respect to the total weight of the solid dosage form.

It has also been found that the manufacturing process is improved by increasing the amount of magnesium stearate. Specifically, the tendency of the final powder mixture to clog the die when processing the powder to solid dosage forms can be reduced.

Thus, an embodiment of the present invention relates to the method as described herein, wherein the final mixture comprises at least 3 wt % magnesium stearate with respect to the total weight of the solid dosage form.

Another embodiment of the present invention relates to the method as described herein, wherein the final mixture comprises 3-5 wt % magnesium stearate with respect to the total weight of the solid dosage form.

However, introduction of lubricants in the melatonin formulation may cause lower wettability and slower disintegration time of the solid dosage form. Higher amounts of lubricants will exacerbate these effects. However, by avoiding extended blending of lubricants, the “over-lubrication” effect can be diminished. Moreover, it has been found that inclusion of disintegrant(s) can offset the negative impact of lubricants on disintegration time and still yield rapid release of melatonin even at high compression force.

Especially for variants of the method wherein solid dosage forms with high amounts of lubricant(s), such as >1 wt %, is produced it is advantageous to reduce the blending time of the lubricant(s).

Thus, an embodiment of the present invention relates to the method as described herein, wherein the third adding step is followed by a second blending of the final mixture.

Another embodiment of the present invention relates to the method as described herein, wherein the second blending is performed for less than 120 seconds, such as less than 90 seconds, such as less than 80 seconds, such as less than 70 seconds, preferably for approximately 60 seconds.

A still further embodiment of the present invention relates to the method as described herein, wherein the second blending is performed at less than 120 rpm, such as at less than 100 rpm, such as at less than 80 rpm, preferably approximately 60 rpm.

Ingredients may be sieved into the mixing container to remove large particulate matter and facilitate uniform distribution of content throughout the powder blend. Sieving is achieved by passing the ingredients through a screen with a defined mesh size. Such screens suitable for industrial use is known to a person skilled in the art.

Therefore, an embodiment of the present invention relates to the method as described herein, wherein the first adding step comprises sieving the one or more fillers through a first screen, sieving the optionally one or more glidants through a second screen, and sieving the optionally one or more disintegrants through a third screen.

Another embodiment of the present invention relates to the method as described herein, wherein the second adding step comprises sieving the second amount of microcrystalline cellulose through a pre-mixing screen, sieving the optionally one or more fillers through a first screen, sieving the optionally one or more glidants through a second screen, and sieving the optionally one or more disintegrants through a third screen.

A further embodiment of the present invention relates to the method as described herein, wherein the third adding step comprises sieving magnesium stearate through a fourth screen.

The mesh size of the screens can be selected to effectively remove particulate matter that impair uniform distribution of content in the powder blend. In general, smaller mesh sizes improves uniform distribution of content but also increase processing time. The method presented herein enables large scale production of solid dosage forms of melatonin without compromising the quality, such as uniformity of content in the solid dosage forms.

Therefore, an embodiment of the present invention relates to the method as described herein, wherein the first screen has a mesh size in the range of 0.8-1.2 mm, the second screen has a mesh size in the range of 2.0-3.0 mm, and/or the third screen has a mesh size in the range of 0.8-1.2 mm.

Another embodiment of the present invention relates to the method as described herein, wherein the fourth screen has a mesh size in the range of 0.8-1.2 mm, preferably approximately 1 mm.

The method described herein comprises a first and a second adding step wherein first and second components, respectively, are added to the mixing container. In variants of the method the order in which the ingredients are mixed are further defined. For example, a preferred order of mixing include only filler as first components and the remaining ingredients as second components. It may also be preferable to add glidant(s) immediately after the stepwise addition of micronized melatonin and MCC.

Therefore, an embodiment of the present invention relates to the method as described herein, wherein the second components comprise one or more glidants.

Another embodiment of the present invention relates to the method as described herein, wherein the second adding step comprises adding the one or more glidants before adding the remaining second components.

A further embodiment of the present invention relates to the method as described herein, wherein the second components comprise one or more disintegrants.

A still further embodiment of the present invention relates to the method as described herein, wherein the second adding step comprises mixing the second amount of microcrystalline cellulose and the one or more disintegrants and sieving them into the mixing container together.

Yet another embodiment of the present invention relates to the method as described herein, wherein the first components comprise one or more fillers.

Another embodiment of the present invention relates to the method as described herein, wherein the first components comprise a filler.

A further embodiment of the present invention relates to the method as described herein, wherein the second components comprise a second amount of microcrystalline cellulose, a glidant and a disintegrant.

It has been found that the method described herein does not require the use of multiple fillers, glidants, disintegrants and/or lubricants to enable production of the rapid release melatonin formulation. By reducing the number of ingredients and simplifying the process, production time and cost can be lowered.

Thus, a preferred embodiment of the present invention relates to the method as described herein, said method comprising the steps of:
i) a first adding step comprising adding first components comprising a filler into a mixing container to form a first mixture,
ii) a stepwise adding step comprising adding providing a first amount of microcrystalline cellulose and a total amount of micronized melatonin, and adding said first amount of microcrystalline cellulose and said total amount of micronized melatonin into the mixing container stepwise in multiple portions,
iii) a second adding step comprising adding second components comprising a second amount of microcrystalline cellulose, a glidant and a disintegrant into the mixing container to form a second mixture,
iv) a third adding step comprising adding magnesium stearate into the mixing container to form a final mixture, and
v) a processing step comprising processing the final mixture to a solid dosage form.

Certain combinations of ingredients and amounts thereof have been demonstrated to produce solid dosage forms with favourable characteristics, such as fast disintegration time, low friability, and high resistance to crushing.

Thus, an embodiment of the present invention relates to the method as described herein, wherein the filler is mannitol, the glidant is colloidal anhydrous silica, and the disintegrant is croscarmellose sodium.

Another embodiment of the present invention relates to the method as described herein, wherein the final mixture comprises:
0.2-3 wt % micronized melatonin,
25-30 wt % microcrystalline cellulose,
60-70 wt % mannitol,
0.4-0.6 wt % colloidal anhydrous silica,
1-3 wt % croscarmellose sodium, and
at least 3 wt % magnesium stearate,
   wherein wt % is with respect to the total weight of the solid dosage form.

Addition and mixing of the ingredients of the melatonin formulation is performed in a mixing container. Any type of mixing container suitable for large scale production can be used in the method. A person skilled in the art is aware of the type of mixing containers that can be used. Typical mixing containers include, but are not limited to, “V” blenders, oblicone blenders, container blenders, tumbling blenders, and agitated powder blenders. For the present method is has been favourable to use a high-shear mixer. The ingredients are added to the mixing container in consecutive steps.

Thus, an embodiment of the present invention relates to the method as described herein, wherein the mixing container is selected a high-shear mixer.

Another embodiment of the present invention relates to the method as described herein, wherein said steps i) through v) are consecutive steps.

The ingredients of the melatonin formulation are provided in dry form, i.e. substantially free from moisture. Preferably, the ingredients are provided as powders.

Accordingly, an embodiment of the present invention relates to the method as described herein, wherein the micronized melatonin, the microcrystalline cellulose and the one or more fillers, glidants and disintegrants are in dry form, such as powders.

The melatonin formulation may be manufactured as any type of solid dosage form suitable for oral administration to an individual. Examples of solid dosage forms include, but are not limited to, tablets, pills, lozenges, capsules, and pastilles.

Thus, an embodiment of the present invention relates to the method as described herein, wherein the solid dosage form is selected from the group consisting of a tablet, pill, lozenge, capsule, and pastille, preferably a tablet.

Another embodiment of the present invention relates to the method as described herein, wherein the solid dosage form is a tablet.

The unit dose of the solid dosage form may comprise between 0.2 mg and 10 mg melatonin. Preferably, the unit dose of the solid dosage form comprises between 0.5 mg and 5 mg melatonin. Solid dosage form may be provided with 0.5 mg, 1 mg, 2 mg, 3 mg, 4 mg or 5 mg melatonin. The solid dosage form have a weight in the range of 50 mg to 300 mg, preferably approximately 200 mg.

Thus, an embodiment of the present invention relates to the method as described herein, wherein the solid dosage form has a weight of approximately 200 mg. Another embodiment of the present invention relates to the method as described herein, wherein the solid dosage forms comprise 0.5 mg, 1 mg, 2 mg, 3 mg, 4 mg or 5 mg melatonin.

Solid dosage forms, such as tablets, are directly compressed from the final powder mixture. This is achieved by filling a die mould followed by compression and ejection of the solid dosage form. Compression may be accomplished by machines including, but not limited to, a single-punch machine (stamping press) or by a multi-station machine (rotary press).

Thus, an embodiment of the present invention relates to the method as described herein, wherein the solid dosage form is a tablet, and the final mixture is tabletted by direct compression.

High compression force improves the stability of solid dosage form but also typically prolongs the release profile of the active ingredient. The method described herein allows solid dosage forms to be manufactured at high compression force and still display a fast release profile.

An embodiment of the present invention relates to the method as described herein, wherein the compression force is in the range of 5-15 kN, preferably in the range of 5-10 kN. A further embodiment relates to the method as described herein, wherein the compression force is in the range of 5-8 kN, preferably approximately 6 kN.

In some variants, the solid dosage form may be provided with a coating. Such a coating is applied in a processing step subsequent to formation of the solid dosage form. Thus, an embodiment of the present invention relates to the method as described herein, wherein the method comprises a further step of coating the solid dosage form.

The coating may be designed to serve a specific purpose, such as sugar coatings, film coatings, gelatin coatings or enteric coatings. Sugar coatings improve the organoleptic properties of solid dosage forms, while film coatings and enteric coatings may be used to improve stability and protect the active ingredient from stomach acid, respectively. An embodiment of the present invention relates to the method as described herein, wherein the coating is selected from the group consisting of sugar coatings, film coatings, gelatin coatings and enteric coatings. Another embodiment of the present invention relates to the method as described herein, wherein the coating comprises one or more ingredients selected from the group consisting of polysaccharides, sucrose, gelatin, hydroxypropyl methylcellulose, glycerin and/or sorbitol.

The rapid release melatonin formulation described herein can be prepared by a method as described herein. The method is suitable for large scale manufacture and produces high quality solid dosages forms with uniform distribution of melatonin, even at low unit dose.

Therefore, an aspect of the present invention relates to a rapid release melatonin formulation obtainable by a method as described herein.

Another aspect of the present invention relates to a rapid release melatonin formulation comprising:
0.2-3 wt % micronized melatonin,
20-35 wt % microcrystalline cellulose,
65-75 wt % of one or more fillers,
0.2-0.8 wt % of one or more glidants,
1-3 wt % of one or more disintegrants, and
at least 3 wt % magnesium stearate,
   wherein the formulation is provided as solid dosage form and wt % is with respect to the total weight of the formulation.

An embodiment of the present invention relates to the rapid release melatonin formulation as described herein, wherein the micronized melatonin has a D90 value of 30 μm or less, preferably 20 μm or less, more preferably 10 μm or less.

The melatonin formulation is not limited to any specific filler(s), glidant(s) or disintegrant(s). Common excipients known to be suitable for production of solid dosage forms, such as tablets, may be included in the melatonin formulation.

Thus, an embodiment of the present invention relates to the rapid release melatonin formulation as described herein, wherein the one or more fillers are selected from the group consisting of mannitol, sorbitol, xylitol, dextrose, sucrose, lactose, polyols, calcium carbonate, calcium sulphate, magnesium oxide, magnesium carbonate, maltodextrin, polymethacrylates, potassium chloride, kaolin, dicalcium phosphate dihydrate, starches, and combinations thereof, preferably mannitol.

A preferred embodiment of the present invention relates to the rapid release melatonin formulation as described herein, wherein the filler is mannitol.

A further embodiment of the present invention relates to the rapid release melatonin formulation as described herein, wherein the one or more glidants are selected from the group consisting of colloidal anhydrous silica, sodium stearyl fumarate, magnesium trisilicate, talc, tribasic calcium phosphate and combinations thereof, preferably colloidal anhydrous silica.

Another preferred embodiment of the present invention relates to the rapid release melatonin formulation as described herein, wherein the glidant is colloidal anhydrous silica.

Yet another embodiment of the present invention relates to the rapid release melatonin formulation as described herein, wherein the one or more disintegrants are selected from the group consisting of croscarmellose sodium, crospovidone, alginic acid, carboxymethylcellulose calcium, carboxymethylcellulose sodium, sodium starch glycolate, carboxymethyl starch, guar gum, magnesium aluminium silicate, methyl cellulose, polacrilin potassium, pregelatinized starch, sodium alginate and combinations thereof, preferably croscarmellose sodium.

Still another preferred embodiment of the present invention relates to the rapid release melatonin formulation as described herein, wherein the disintegrant is croscarmellose sodium.

Certain combinations of ingredients have proven to yield favourable solid dosage forms with desired properties, such as fast disintegration time and high stability.

Therefore, an embodiment of the present invention relates to the rapid release melatonin formulation as described herein, wherein the filler is mannitol, the glidant is colloidal anhydrous silica, and the disintegrant is croscarmellose sodium.

Another embodiment of the present invention relates to the rapid release melatonin formulation as described herein, wherein the formulation comprises:
0.2-3 wt % micronized melatonin,
25-30 wt % microcrystalline cellulose,
60-70 wt % mannitol,
0.4-0.6 wt % colloidal anhydrous silica,
1-3 wt % croscarmellose sodium, and
at least 3 wt % magnesium stearate.

For some purpose it may be desirable to add small amounts of additional ingredients to improve the melatonin formulation and the final solid dosage form. Therefore, an embodiment of the present invention relates to the rapid release melatonin formulation as described herein, wherein the formulation further comprises a lubricant selected from group consisting of calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl furmarate, stearic acid, talc and zinc stearate.

It may also be desirable to add one or more flavouring agents to improve the organoleptic experience of the consumer. Such flavouring agents will only be added in small amounts that does not alter the physical or pharmacokinetic properties of the solid dosage form.

Accordingly, an embodiment of the present invention relates to the rapid release melatonin formulation as described herein, wherein the formulation further comprises one or more flavouring agents.

Another embodiment of the present invention relates to the rapid release melatonin formulation as described herein, wherein the one or more flavouring agents are selected from the group consisting of maltol, vanillin, ethyl vanillin, menthol, citric acid, fumaric acid, ethyl maltol, tartaric acid, and combinations thereof.

The rapid release may be produced at different strength, i.e. unit dose, that are suitable for different situations and individuals. Especially, solid dosage forms of low strength may be particularly suited for administration to children or adolescents.

Another embodiment of the present invention relates to the rapid release melatonin formulation as described herein, wherein the formulation comprises:
0.25-2.5 wt % micronized melatonin,
28.5 wt % microcrystalline cellulose,
63.5-65.75 wt % mannitol,
0.5 wt % colloidal anhydrous silica,
2 wt % croscarmellose sodium, and
3 wt % magnesium stearate.

A further embodiment of the present invention relates to the rapid release melatonin formulation as described herein, wherein the formulation comprises 0.25 wt %, 0.5 wt %, 1 wt %, 1.5 wt %, 2 wt % or 2.5 wt % micronized melatonin.

Yet another embodiment of the present invention relates to the rapid release melatonin formulation as described herein, wherein the formulation comprises:
0.25 wt % micronized melatonin,
28.5 wt % microcrystalline cellulose,
65.75 wt % mannitol,
0.5 wt % colloidal anhydrous silica,
3 wt % magnesium stearate.

2 wt % croscarmellose sodium, and

A still further embodiment of the present invention relates to the rapid release melatonin formulation as described herein, wherein the formulation comprises:
about 0.5 wt % micronized melatonin,
about 28.5 wt % microcrystalline cellulose,
about 65.5 wt % mannitol,
about 0.5 wt % colloidal anhydrous silica,
about 2 wt % croscarmellose sodium, and
about 3 wt % magnesium stearate.

Yet another embodiment of the present invention relates to the rapid release melatonin formulation as described herein, wherein the formulation comprises:
about 2.5 wt % micronized melatonin,
about 28.5 wt % microcrystalline cellulose,
about 63.5 wt % mannitol,
about 0.5 wt % colloidal anhydrous silica,
about 2 wt % croscarmellose sodium, and
about 3 wt % magnesium stearate.

The melatonin formulation may be provided as any type of solid dosage form that is suitable for oral administration.

Therefore, an embodiment of the present invention relates to the rapid release melatonin formulation as described herein, wherein the solid dosage form is selected from the group consisting of a tablet, pill, lozenge, capsule, and pastille, preferably a tablet.

Another embodiment of the present invention relates to the rapid release melatonin formulation as described herein, wherein the solid dosage form is a tablet.

A further embodiment of the present invention relates to the rapid release melatonin formulation as described herein, wherein the solid dosage form is an immediate release tablet.

The rapid release melatonin tablet has a fast disintegration time which promotes its fast bioavailability upon ingestion. Consequently, it is suitable, amongst others, in situations where a quick sleep-inducing effect is preferred. Disintegration time of tablets is defined in the European Pharmacopoeia 10^{th} Edition, 2.9.1 Disintegration of Tablets and Capsules (Ph.Eur.2.9.1) and is herein measured on a tablet disintegration tester of the brand Erweka ZT53. The fast disintegration time is evidenced herein and does not compromise other physical properties of the tablet.

Thus, an embodiment of the present invention relates to the rapid release melatonin formulation as described herein, wherein the disintegration time of the tablet is less than 60 seconds, such as less than 55 seconds, such as less than 50 seconds, such as less than 45 seconds, less than 40 seconds.

Another embodiment of the present invention relates to the rapid release melatonin formulation as described herein, wherein the disintegration time is defined in accordance with European Pharmacopoeia 10^{th} Edition, 2.9.1 Disintegration of Tablets and Capsules (Ph.Eur.2.9.1).

A further embodiment of the present invention relates to the rapid release melatonin formulation as described herein, wherein the disintegration time is measured on a tablet disintegration tester of the brand Erweka ZT53.

Yet another embodiment of the present invention relates to the rapid release melatonin formulation as described herein, wherein at least 90%, preferably at least 95%, of the nominal content of the tablet is released within 15 minutes.

A still further embodiment of the present invention relates to the rapid release melatonin formulation as described herein, wherein at least 95%, such as at least 96%, such as at least 97%, of the nominal content of the rapid release melatonin formulation is released within 15 minutes as determined in accordance with European Pharmacopoeia 10^{th} Edition, 2.9.3 Dissolution test for solid dosage forms. Preferably, the rapid release melatonin formulation is a tablet.

The method disclosed herein facilitates that the active ingredient (melatonin) is uniformly distributed across the formulation thereby ensuring a defined amount of active ingredient in each dosage unit. This is important to certify that the recipient of the dosage unit receives an intended amount of active ingredient upon ingestion.

Consistency of the dosage units is assessed by the acceptance value (AV), which is a threshold set to evaluate the dosage units against a reference value. The method of determining the content uniformity and the acceptance value is described in European Pharmacopoeia 10^{th} Edition, 2.9.40 Uniformity of dosage units.

Thus, an embodiment of the present invention relates to the rapid release melatonin formulation as described herein, wherein the content uniformity of the rapid release melatonin formulation is at least 95%, such as at least 96%, such as at least 97%, such as at least 98%, such as at least 99%, as determined in accordance with European Pharmacopoeia 10^{th} Edition, 2.9.40 Uniformity of dosage units. Preferably, the rapid release melatonin formulation is in the form of a tablet.

Another embodiment of the present invention relates to the rapid release melatonin formulation as described herein, wherein the acceptance value (AV) of the rapid release melatonin formulation is less than 10, such as less than 8, such as less than 7, such as less than 6, as determined in accordance with European Pharmacopoeia 10^{th} Edition, 2.9.40 Uniformity of dosage units. Preferably, the rapid release melatonin formulation is in the form of a tablet.

Physical properties of the tablet are tested during manufacture by manual and automated sampling or by standard in-process control (IPC) testing. Among the parameters tested are friability, resistance to crushing and tensile strength. The tablets presented herein fulfil all the standard IPC requirements.

Therefore, an embodiment of the present invention relates to the rapid release melatonin formulation as described herein, wherein the tablet has a friability of a less than 1%, such as less than 0.7%, such as less than 0.5%.

Another embodiment of the present invention relates to the rapid release melatonin formulation as described herein, wherein the tablet has a friability in the range of 0.2-0.5%.

A further embodiment of the present invention relates to the rapid release melatonin formulation as described herein, wherein the tablet has a resistance to crushing in the range of 30-100 N, preferably in the range of 40-80 N.

Yet another embodiment of the present invention relates to the rapid release melatonin formulation as described herein, wherein the tablet has a tensile strength in the range of 0.5-3.0 MPa, such as in the range of 1-2 MPa.

Particularly favourable rapid release melatonin formulations have been identified and provided in dosage units suitable for administration to patients. The need of the patient may vary and the strength of the rapid release melatonin formulation may therefore be altered accordingly.

Thus, an embodiment of the present invention relates to the rapid release melatonin formulation as described herein, wherein the tablet comprises about 0.5-5 mg micronized melatonin.

Another embodiment of the present invention relates to the rapid release melatonin formulation as described herein, wherein the tablet comprises about 0.5 mg, 1 mg, 1.5 mg, 2 mg, 2.5 mg, 3 mg, 3.5 mg, 4 mg, 4.5 mg or 5 mg micronized melatonin, preferably 0.5 mg, 1 mg, 3 mg, or 5 mg micronized melatonin.

The rapid release melatonin formulation described herein is useful for treatment of sleep disorders, such as insomnia. Many variants of insomnia exist including, but not limited to, sleep maintenance insomnia, terminal insomnia, sleep onset insomnia, and psychophysiological insomnia. However, the rapid release melatonin formulation may also be used in the treatment of disorder associated with the circadian rhythm including, but not limited to, jet lag, shift work sleep disorder, delayed sleep phase disorder (DSPS) and non-24-hour sleep wake disorder.

Other patient groups that may benefit from the rapid release melatonin formulation described herein are patients experiencing sleep disorders derived from psychiatric conditions and/or neurological disease. These patients may suffer from attention deficit hyperactivity disorder (ADHD), autism spectrum disorder (ASD), dementia or parkinsonism. Moreover, sleep disorders often occur in combination with mental disorders, such as psychoses or mood and anxiety disorders, or in conjunction with medical disorders, such as chronic obstructive pulmonary disease and nocturnal cardiac ischemia.

Accordingly, an aspect of the present invention relates to a rapid release melatonin formulation as described herein for use as a medicament.

Another aspect of the present invention relates to a rapid release melatonin formulation as described herein for use in the treatment of sleep disorders.

An embodiment of the present invention relates to the rapid release melatonin formulation for use as described herein, wherein the sleep disorders are selected from the group consisting of insomnia, sleep disorders associated with the circadian rhythm, delayed sleep phase disorder (DSPS), jet lag, sleep disorders associated with a psychiatric condition, sleep disorders associated with neurological disease, sleep disorders associated with a mental condition, sleep disorders associated with a medical disorder.

Another embodiment of the present invention relates to the rapid release melatonin formulation for use as described herein, wherein the sleep disorders is insomnia due to ADHD.

A further embodiment of the present invention relates to the rapid release melatonin formulation for use as described herein, wherein the rapid release melatonin formulation is administered to a child or adolescent.

A still further embodiment of the present invention relates to the rapid release melatonin formulation for use as described herein, wherein the rapid release melatonin formulation is administered to a child or adolescent with ADHD or a developmental disorder.

Yet another embodiment of the present invention relates to the rapid release melatonin formulation for use as described herein, wherein the rapid release melatonin formulation is administered to an adult aged 50 years or older, such as 55 years or older, such as 60 years or older, such as 70 years or older, such as 80 years or older.

Still another embodiment of the present invention relates to the rapid release melatonin formulation for use as described herein, wherein the rapid release melatonin formulation is administered to an individual that has discontinued the use of a benzodiazepine or non-benzodiazepine hypnotic.

A preferred embodiment of the present invention relates to the rapid release melatonin formulation for use as described herein, wherein the sleep disorder is insomnia.

The rapid release melatonin formulation described herein is designed for oral ingestion. Thus, an embodiment of the present invention relates to the rapid release melatonin formulation for use as described herein, wherein the route of administration is orally. The melatonin formulation may be swallowed, orally disintegrated or chewed, preferably swallowed.

The rapid release melatonin formulation may conveniently be provided to the end-user as a kit of parts with multiple solid dosage forms for continuous use. The kit will typically hold information on the use of the content of the kit. The solid dosage forms can be practically packaged as is common routine for solid dosage forms and would be known by the person skilled in the art. Such packaging is typically a bottle, such as a plastic bottle.

Thus, an aspect of the present invention relates to a kit of parts comprising:
a plurality of solid dosage forms comprising a rapid release melatonin formulation as described herein, and
optionally, instructions for use.

An embodiment of the present invention relates to the kit of parts as described herein, wherein the plurality of solid dosage forms is provided in a bottle, a blister package or in aluminium pouches. Preferably, the solid dosage forms are packaged in a bottle, preferably a plastic bottle.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the dry composition and all its features, which may readily be part of the final dry composition obtained by the method as described herein. Embodiments and features of the present invention are also outlined in the following items.

### Items

X1. A method for manufacture of a rapid release melatonin formulation in solid dosage form, said method comprising:
i) a first adding step comprising adding first components into a mixing container to form a first mixture, wherein the first components comprise one or more fillers and optionally one or more glidants and/or disintegrants,
ii) a stepwise adding step comprising providing a first amount of microcrystalline cellulose and a total amount of micronized melatonin, and adding said first amount of microcrystalline cellulose and said total amount of micronized melatonin into the mixing container stepwise in multiple portions,
iii) a second adding step comprising adding second components into the mixing container to form a second mixture, wherein the second components comprise a second amount of microcrystalline cellulose and optionally one or more fillers, glidants and/or disintegrants, and wherein the second mixture comprises micronized melatonin, microcrystalline cellulose, filler, glidant and disintegrant
iv) a third adding step comprising adding magnesium stearate into the mixing container to form a final mixture, and
v) a processing step comprising processing the final mixture to a solid dosage form,
   wherein at least one of the first and/or second adding step comprises adding one or more glidants, and
   wherein at least one of the first and/or second adding step comprises adding one or more disintegrants.

X2. The method according to item X1, wherein the ratio between said first amount of microcrystalline cellulose and said one or more fillers of the first components is in the range of 1:8 to 1:12 wt %/wt %, preferably approximately 1:10 wt %/wt %, with respect to the total weight of the solid dosage form.

X3. The method according to any one of items X1 or X2, wherein the ratio between said first amount of microcrystalline cellulose and said second amount of microcrystalline cellulose is in the range of 1:4 to 1:10 wt %/wt %, preferably approximately 1:5 wt %/wt %, with respect to the total weight of microcrystalline cellulose in the solid dosage form.

X4. The method according to any one of the preceding items, wherein each portion of the stepwise adding step comprises a fraction of said first amount of microcrystalline cellulose and a fraction of said total amount of micronized melatonin.

X5. The method according to any one of the preceding items, wherein each portion of the stepwise adding step comprises between 10-50 wt % of said first amount of microcrystalline cellulose, such as 15-40 wt % of said first amount of microcrystalline cellulose, such as 15-25 wt % of said first amount of microcrystalline cellulose, preferably approximately 20 wt % of said first amount of microcrystalline cellulose.

X6. The method according to any one of the preceding items, wherein each portion of the stepwise adding step comprises between 10-50 wt % of said total amount of micronized melatonin, such as 15-40 wt % of said total amount of micronized melatonin, such as 15-25 wt % of said total amount of micronized melatonin, preferably approximately 20 wt % of said total amount of micronized melatonin.

X7. The method according to any one of the preceding items, wherein said multiple portions are three portions, four portions, five portions, six portions, seven portions, or eight portions, preferably five portions.

X8. The method according to any one of the preceding items, wherein the stepwise adding step comprises sieving the total amount of micronized melatonin and the first amount of microcrystalline cellulose through a pre-mixing screen.

X9. The method according to item X8, wherein the pre-mixing screen has a mesh size in the range of 0.8-1.2 mm, preferably approximately 1 mm.

X10. The method according to any one of the preceding items, wherein each portion of the stepwise adding step comprises a fraction of said first amount of microcrystalline cellulose and a fraction of said total amount of micronized melatonin that are sieved together through the pre-mixing screen.

X11. The method according to any one of the preceding items, wherein the stepwise adding step comprises providing the first amount of microcrystalline cellulose and the total amount of micronized melatonin as a pre-mix blend.

X12. The method according to item X11, wherein each portion of the stepwise adding step comprises between 10-35 wt % of said pre-mix blend, such as 15-25 wt % of said pre-mix blend, preferably approximately 20 wt % of said pre-mix blend.

X13. The method according to any one of the preceding items, wherein the one or more fillers are selected from the group consisting of mannitol, sorbitol, xylitol, dextrose, sucrose, lactose, polyols, calcium carbonate, calcium sulphate, magnesium oxide, magnesium carbonate, maltodextrin, polymethacrylates, potassium chloride, kaolin, dicalcium phosphate dihydrate, starches, and combinations thereof, preferably mannitol.

X14. The method according to any one of the preceding items, wherein the one or more glidants are selected from the group consisting of colloidal anhydrous silica, sodium stearyl fumarate, magnesium trisilicate, talc, tribasic calcium phosphate and combinations thereof, preferably colloidal anhydrous silica.

X15. The method according to any one of the preceding items, wherein the one or more disintegrants are selected from the group consisting of croscarmellose sodium, crospovidone, alginic acid, carboxymethylcellulose calcium, carboxymethylcellulose sodium, sodium starch glycolate, carboxymethyl starch, guar gum, magnesium aluminium silicate, methyl cellulose, polacrilin potassium, pregelatinized starch, sodium alginate and combinations thereof, preferably croscarmellose sodium.

X16. The method according to any one of the preceding items, wherein the final mixture comprises 0.2-3 wt % micronized melatonin with respect to the total weight of the solid dosage form.

X17. The method according to any one of the preceding items, wherein the final mixture comprises 20-35 wt % microcrystalline cellulose with respect to the total weight of the solid dosage form.

X18. The method according to any one of the preceding items, wherein the final mixture comprises 65-75 wt % of one or more fillers with respect to the total weight of the solid dosage form.

X19. The method according to any one of the preceding items, wherein the final mixture comprises 0.2-0.8 wt % of one or more glidants with respect to the total weight of the solid dosage form.

X20. The method according to any one of the preceding items, wherein the final mixture comprises 1-3 wt % of one or more disintegrants with respect to the total weight of the solid dosage form.

X21. The method according to any one of the preceding items, wherein the final mixture comprises at least 3 wt % magnesium stearate with respect to the total weight of the solid dosage form.

X22. The method according to any one of the preceding items, wherein the first mixture comprises at least 80 wt % of the one or more fillers, such as at least 85 wt %, such as at least 90 wt %, such as at least 95 wt %, with respect to the total weight of the one or more fillers in the solid dosage form.

X23. The method according to any one of the preceding items, wherein the second adding step is followed by a first blending of the second mixture.

X24. The method according to item X23, wherein the first blending is performed for a duration of 40-240 seconds, such as 60-180 seconds, such as 100-140 seconds, preferably for approximately 120 seconds.

X25. The method according to any one of items X23 or X24, wherein the first blending is performed at less than 120 rpm, such as at less than 100 rpm, such as at less than 80 rpm, preferably at less than 60 rpm.

X26. The method according to any one of the preceding items, wherein the third adding step is followed by a second blending of the final mixture.

X27. The method according to item X26, wherein the second blending is performed for less than 120 seconds, such as less than 90 seconds, such as less than 80 seconds, such as less than 70 seconds, preferably for approximately 60 seconds.

X28. The method according to any one of items X26 or X27, wherein the second blending is performed at less than 120 rpm, such as at less than 100 rpm, such as at less than 80 rpm, preferably approximately 60 rpm.

X29. The method according to any one of the preceding items, wherein the first adding step comprises sieving the one or more fillers through a first screen, sieving the optionally one or more glidants through a second screen, and sieving the optionally one or more disintegrants through a third screen.

X30. The method according to any one of the preceding items, wherein the second adding step comprises sieving the second amount of microcrystalline cellulose through a pre-mixing screen, sieving the optionally one or more fillers through a first screen, sieving the optionally one or more glidants through a second screen, and sieving the optionally one or more disintegrants through a third screen.

X31. The method according to any one of items X29 or X30, wherein the first screen has a mesh size in the range of 0.8-1.2 mm, the second screen has a mesh size in the range of 2.0-3.0 mm, and/or the third screen has a mesh size in the range of 0.8-1.2 mm.

X32. The method according to any one of the preceding items, wherein the third adding step comprises sieving magnesium stearate through a fourth screen.

X33. The method according to item X32, wherein the fourth screen has a mesh size in the range of 0.8-1.2 mm, preferably approximately 1 mm.

X34. The method according to any one of the preceding items, wherein the second components comprise one or more glidants.

X35. The method according to item X34, wherein the second adding step comprises adding the one or more glidants before adding the remaining second components.

X36. The method according to any one of the preceding items, wherein the second components comprise one or more disintegrants.

X37. The method according to item X36, wherein the second adding step comprises mixing the second amount of microcrystalline cellulose and the one or more disintegrants and sieving them into the mixing container together.

X38. The method according to any one of the preceding items, wherein the first components comprise one or more fillers.

X39. The method according to any one of the preceding items, wherein the first components comprise a filler.

X40. The method according to any one of the preceding items, wherein the second components comprise a second amount of microcrystalline cellulose, a glidant and a disintegrant.

X41. The method according to any one of the preceding items, said method comprising the steps of:
i) a first adding step comprising adding first components comprising a filler into a mixing container to form a first mixture,
ii) a stepwise adding step comprising adding providing a first amount of microcrystalline cellulose and a total amount of micronized melatonin, and adding said first amount of microcrystalline cellulose and said total amount of micronized melatonin into the mixing container stepwise in multiple portions,
iii) a second adding step comprising adding second components comprising a second amount of microcrystalline cellulose, a glidant and a disintegrant into the mixing container to form a second mixture,
iv) a third adding step comprising adding magnesium stearate into the mixing container to form a final mixture, and
v) a processing step comprising processing the final mixture to a solid dosage form.

X42. The method according to any one of the preceding items, wherein the filler is mannitol, the glidant is colloidal anhydrous silica, and the disintegrant is croscarmellose sodium.

X43. The method according to any one of the preceding items, wherein the final mixture comprises:
0.2-3 wt % micronized melatonin,
25-30 wt % microcrystalline cellulose,
60-70 wt % mannitol,
0.4-0.6 wt % colloidal anhydrous silica,
1-3 wt % croscarmellose sodium, and
at least 3 wt % magnesium stearate,
   wherein wt % is with respect to the total weight of the solid dosage form.

X44. The method according to any one of the preceding items, wherein said steps i) through v) are consecutive steps.

X45. The method according to any one of the preceding items, wherein the mixing container is selected a high-shear mixer.

X46. The method according to any one of the preceding items, wherein the micronized melatonin, the microcrystalline cellulose and the one or more fillers, glidants and disintegrants are in dry form, such as powders.

X47. The method according to any one of the preceding items, wherein the solid dosage form is selected from the group consisting of a tablet, pill, lozenge, capsule, and pastille, preferably a tablet.

X48. The method according to any one of the preceding items, wherein the solid dosage form is a tablet, and the final mixture is tabletted by direct compression.

X49. The method according to item X46, wherein the compression force is in the range of 5-15 kN, preferably in the range of 5-10 kN.

X50. The method according to any one of items X47-X49, wherein the content uniformity of the tablet is at least about 95%, such as at least about 96%, such as at least about 97%, such as at least about 98%, such as at least about 99%, as determined in accordance with European Pharmacopoeia 10^{th} Edition, 2.9.40 Uniformity of dosage units.

X51. The method according to item X50, wherein the acceptance value (AV) of the rapid release melatonin formulation is less than about 10, such as less than about 8, such as less than about 7, such as less than about 6, as determined in accordance with European Pharmacopoeia 10^{th} Edition, 2.9.40 Uniformity of dosage units.

X52. The method according to any one of items X47-X51, wherein at least about 90%, preferably at least about 95%, of the nominal content of the tablet is released within about 15 minutes.

X53. The method according to any one of the preceding items, wherein the micronized melatonin comprises a population of melatonin particles with a particle size (D90) of about 30 μm or less when measured using laser diffraction.

X54. The method according to item X53, wherein the particle size is determined using a Malvern Mastersizer 2000 from Malvern Instruments.

Y1. A rapid release melatonin formulation comprising:
0.2-3 wt % micronized melatonin,
20-35 wt % microcrystalline cellulose,
65-75 wt % of one or more fillers,
0.2-0.8 wt % of one or more glidants,
1-3 wt % of one or more disintegrants, and
at least 3 wt % magnesium stearate,
   wherein the formulation is provided as solid dosage form and wt % is with respect to the total weight of the formulation.

Y2. The rapid release melatonin formulation according to item Y1, wherein the one or more fillers are selected from the group consisting of mannitol, sorbitol, xylitol, dextrose, sucrose, lactose, polyols, calcium carbonate, calcium sulphate, magnesium oxide, magnesium carbonate, maltodextrin, polymethacrylates, potassium chloride, kaolin, dicalcium phosphate dihydrate, starches, and combinations thereof, preferably mannitol.

Y3. The rapid release melatonin formulation according to any one of items Y1 or Y2, wherein the one or more glidants are selected from the group consisting of colloidal anhydrous silica, sodium stearyl fumarate, magnesium trisilicate, talc, tribasic calcium phosphate and combinations thereof, preferably colloidal anhydrous silica.

Y4. The rapid release melatonin formulation according to any one of items Y1-Y3, wherein the one or more disintegrants are selected from the group consisting of croscarmellose sodium, crospovidone, alginic acid, carboxymethylcellulose calcium, carboxymethylcellulose sodium, sodium starch glycolate, carboxymethyl starch, guar gum, magnesium aluminium silicate, methyl cellulose, polacrilin potassium, pregelatinized starch, sodium alginate and combinations thereof, preferably croscarmellose sodium.

Y5. The rapid release melatonin formulation according to any one of items Y1-Y4, wherein the filler is mannitol, the glidant is colloidal anhydrous silica, and the disintegrant is croscarmellose sodium.

Y6. The rapid release melatonin formulation according to any one of items Y1-Y5, wherein the formulation comprises:
0.2-3 wt % micronized melatonin,
25-30 wt % microcrystalline cellulose,
60-70 wt % mannitol,
0.4-0.6 wt % colloidal anhydrous silica,
1-3 wt % croscarmellose sodium, and
at least 3 wt % magnesium stearate.

Y7. The rapid release melatonin formulation according to any one of items Y1-Y6, wherein the formulation further comprises a lubricant selected from group consisting of calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl furmarate, stearic acid, talc and zinc stearate.

Y8. The rapid release melatonin formulation according to any one of items Y1-Y7, wherein the formulation further comprises one or more flavouring agents.

Y9. The rapid release melatonin formulation according to item Y8, wherein the one or more flavouring agents are selected from the group consisting of maltol, vanillin, ethyl vanillin, menthol, citric acid, fumaric acid, ethyl maltol, tartaric acid, and combinations thereof.

Y10. The rapid release melatonin formulation according to any one of items Y1-Y9, wherein the solid dosage form is selected from the group consisting of a tablet, pill, lozenge, capsule, and pastille, preferably a tablet.

Y11. The rapid release melatonin formulation according to item Y10, wherein the solid dosage form is an immediate release tablet.

Y12. The rapid release melatonin formulation according to any one of items Y10 or Y11, wherein the disintegration time of the tablet is less than 60 seconds, such as less than 55 seconds, such as less than 50 seconds, such as less than 45 seconds, less than 40 seconds.

Y13. The rapid release melatonin formulation according to any one of items Y10-Y12, wherein the tablet has a friability of a less than 1%, such as less than 0.7%, such as less than 0.5%.

Y14. The rapid release melatonin formulation according to any one of items Y10-Y13, wherein the tablet has a resistance to crushing in the range of 30-100 N, preferably in the range of 40-80 N.

Y15. The rapid release melatonin formulation according to any one of items Y10-Y14, wherein the tablet has a tensile strength in the range of 0.5-3.0 MPa, such as in the range of 1-2 MPa.

Y16. The rapid release melatonin formulation according to any one of items Y10-Y15, wherein the content uniformity of the tablet is at least about 95%, such as at least about 96%, such as at least about 97%, such as at least about 98%, such as at least about 99%, as determined in accordance with European Pharmacopoeia 10^{th} Edition, 2.9.40 Uniformity of dosage units.

Y17. The rapid release melatonin formulation according to item Y16, wherein the acceptance value (AV) of the tablet is less than about 10, such as less than about 8, such as less than about 7, such as less than about 6, as determined in accordance with European Pharmacopoeia 10^{th} Edition, 2.9.40 Uniformity of dosage units.

Y18. The rapid release melatonin formulation according to any one of items Y10-Y17, wherein at least about 90%, preferably at least about 95%, of the nominal content of the tablet is released within about 15 minutes.

Y19. The rapid release melatonin formulation according to any one of items Y10-Y18, wherein the micronized melatonin comprises a population of melatonin particles with a particle size (D90) of about 30 μm or less when measured using laser diffraction.

Y20. The rapid release melatonin formulation according to item Y19, wherein the particle size is determined using a Malvern Mastersizer 2000 from Malvern Instruments.

Z1. A rapid release melatonin formulation obtainable by a method according to any one of items X1-X54.

W1. A rapid release melatonin formulation according to items Y1-Y20 or Z1 for use as a medicament.

W2. A rapid release melatonin formulation according to items Y1-Y20 or Z1 for use in the treatment of sleep disorders.

W3. The rapid release melatonin formulation for use according to item W2, wherein the sleep disorders are selected from the group consisting of insomnia, sleep disorders associated with the circadian rhythm, delayed sleep phase disorder (DSPS), jet lag, sleep disorders associated with a psychiatric condition, sleep disorders associated with neurological disease, sleep disorders associated with a mental condition, sleep disorders associated with a medical disorder.

U1. Kit of parts comprising:
a plurality of solid dosage forms comprising a rapid release melatonin formulation according to items Y1-Y20 or Z1, and
optionally, instructions for use.

U2. The kit of parts according to U1, wherein the plurality of solid dosage forms is provided in a bottle, a blister package or in aluminium pouches.

The invention will now be described in further details in the following non-limiting examples.

### EXAMPLES

### Example 1: Effect of Using Non-Micronized Melatonin or Micronized Melatonin on the Homogeneity of Tablets

This example investigated if direct compression was a feasible approach to obtain tablets with acceptable uniformity of content of two basic formulations. Thus, tablets with either non-micronized melatonin or micronized melatonin were prepared.

### Method:

Tablets of either 1 mg or 5 mg API (micronized or non-micronized melatonin) were prepared with a composition according to table 1. Batches of approximately 1000 g were manufactured.

TABLE 1

| Strength | 1 mg/tablet | | 5 mg/tablet | |
|---|---|---|---|---|
| Ingredient | mg/tablet | % | mg/tablet | % |
| Melatonin | 1 | 0.5 | 5 | 2.5 |
| Microcrystalline cellulose | 57 | 28.5 | 57 | 28.5 |
| Mannitol | 131 | 65.5 | 127 | 63.5 |
| Silica colloidal anhydrous | 1 | 0.5 | 1 | 0.5 |
| Magnesium stearate | 6 | 3.0 | 6 | 3.0 |
| Croscarmellose sodium | 4 | 2.0 | 4 | 2.0 |
| Total | 200 | 100 | 200 | 100 |

The raw materials were acquired according to table 2.

TABLE 2
Suppliers of ingredients.

| Ingredient | Trade name | Supplier |
|---|---|---|
| Melatonin (non-micronized) | Melatonin F | Flamma S.p.A. |
| Melatonin micronized | Melatonin F micro | Flamma S.p.A. |
| Melatonin micronized | Melatonin (BP) | Swati |
| Microcrystalline cellulose | Avicel PH-102 | Dupont/FMC |
| | | Biopolymer |
| Mannitol | Pearlitol SD100 | Roquette |
| Silica colloidal anhydrous | Aerosil 200 | Evonik |
| Magnesium stearate | Ligamed MF-2-V | Peter Greven |
| Croscarmellose sodium | Ac-Di-Sol | Dupont/FMC |
| | | Biopolymer |

### Micronization

Melatonin from Flamma S.p.A was available in two different particle sizes—one micronized (Melatonin F micro) and one non-micronized (Melatonin F). An additional supplier (Swati) provided micronized melatonin particles of slightly larger size than Flamma S.p.A. To probe additional micronized particle sizes, melatonin was micronized as described below to obtain additionally two particle sizes of melatonin (experiment name 1A and 1C, table 3).

Micronization was performed using a jet mill, placed in a glove box. The jet mill was either equipped with a micronization ring with 3 holes (1.3 mm), micro-mixing ring with 6 holes (2.9 mm) or micro-mixing ring with 5 holes (3.5 mm). One gram of melatonin was micronized with selected settings (ejector pressure and milling pressure) to saturate the surfaces in the mill or clean the mill. The collecting can was then replaced with another collecting can and the micronization continued.

In order to avoid spontaneous and unwanted recrystallization, exposure to elevated humidity at any step was avoided and the micronized substance was transferred to adequate container with active drying agent as soon as possible.

### Mixing of Ingredients

Mixing was performed using a high shear mixer (Diosna 4 litres). Melatonin was handled using stainless steel utensils due to its affinity to plastic. The following protocol was used for mixing the ingredients.
1. Mannitol was sieved through a 1 mm screen into a Diosna vessel 4 liter.
2. Microcrystalline cellulose (MCC, 20%) (A) and melatonin (M) were sieved alternately through a 1 mm screen into the mixing container. The following order of alternating mixing was used: AM AM AM AM AM
3. Silica colloidal anhydrous was sieved through a 2 mm screen into the mixing container.
4. Croscarmellose sodium and the remaining part of MCC (80%) were sieved through a 1 mm screen into the mixing container.
5. Mixing step 1: 300 rpm 2 min
6. Magnesium stearate was sieved through a 1 mm screen into the mixing container.
7. Mixing step 2: 300 rpm 1 min

### Tablet Compression

The powder mixtures were compressed on a Fette 52i tablet press, using four sets of 8 mm circular cupped punches and a 10 mm fill cam. A force feeder was used to facilitate the filling of the dies. Filling depth was adjusted to reach a target tablet weight of 200 mg. The turret speed was 20 rpm and the force feeder speed was 22 rpm. The average tablet weight (n=10) was checked every 5 min.

### Particle Size Distribution

Analysis of particle size distribution was performed using a Malvern Mastersizer 2000 with a measuring cell Hydro 2000 μP. Measurements were performed for micronized melatonin and non-micronized melatonin, respectively, as described below.

### Micronized Samples

Paraffin was used as dispersion agent. Sample was added until the obscuration value was between 3-5. The sample was stirred continuously at 2500 rpm. Sonication of the sample was performed for 10 seconds and particle size analysis was performed after 20 seconds of equilibration to avoid thermal artefacts. Sonication was repeated until the particle size distribution was stable.

### Non-Micronized Samples

Heptane was used as dispersion agent. Sample was added until the obscuration value was between 9-10. The sample was stirred continuously at 2500 rpm. Sonication was performed for 10 minutes in the measuring cell before analysis.

### Uniformity

Analyses of content uniformity were performed on ten samples/units evaluating the relative standard deviation (RSD, %). Assay was carried out by reversed phase liquid chromatography and UV detection. Quantification was made by the use of an external standard. Analysis was conducted in accordance with Ph. Eur.2.9.40.

### Results:

The content uniformity for the two batches with non-micronized melatonin were worse than the batches with micronized melatonin, and RSD was significantly higher. Content and RSD were considered acceptable for all compositions with micronized melatonin (1A-1C, 1E, 5A-5C).

The calculated acceptance value for the compositions was low except for 1D and 5D, i.e. the batches with largest melatonin particle size (see table 3). According to Ph. Eur, acceptable AV for ten tablets is less than 15. As seen in the table, this is readily fulfilled for all samples comprising micronized melatonin.

TABLE 3
The table shows the content uniformity for tablets comprising
melatonin of different particle size distribution and tablet strength.
For the content uniformity, relative standard deviation (RSD) and
acceptance value (AV) according to the European Pharmacopoeia
10^{th} Edition is given.

| | | Melatonin | | | |
|---|---|---|---|---|---|
| | Tablet | particle | Content | | |
| Experiment | strength | size | uniformity | RSD | |
| name | (mg/tablet) | (D90, μm) | (%) | (%) | AV |
| 1A | 1 | 6.0 | 99.11 | 1.13 | 2.7 |
| 1B | 1 | 7.5 | 100.87 | 0.77 | 1.9 |
| 1C | 1 | 9.6 | 100.18 | 1.13 | 2.7 |
| 1D | 1 | 131.8 | 93.57 | 11.39 | 30.5 |
| 1E | 1 | 14.8 | 99.15 | 2.02 | 4.8 |
| 5A | 5 | 6.0 | 99.18 | 2.26 | 5.4 |
| 5B | 5 | 7.5 | 99.74 | 0.58 | 1.4 |
| 5C | 5 | 9.6 | 100.35 | 0.57 | 1.4 |
| 5D | 5 | 131.8 | 100.77 | 4.62 | 11.2 |

### Conclusion:

This example demonstrates that tablets comprising micronized melatonin can be directly compressed to yield tablets of high uniformity of melatonin, whereas melatonin with larger particle size do not ensure homogeneous distribution of content between tablets. Moreover, the process produced tablet of high uniformity even with very reduced mixing times.

### Example 2: Effect of Using Non-Micronized Melatonin or Micronized Melatonin on the Dissolution of Tablets

This example investigated how the dissolution rate of the melatonin tablets was affected by the melatonin particle size.

### Method:

Tablets of either 1 mg or 5 mg API (micronized or non-micronized melatonin) were prepared according to example 1.

### Dissolution Testing

The analysis was carried out with one tablet in 500 mL medium (water) at 37° C. and 50 rpm, n=6. Quantification was made by reversed phase liquid chromatography and UV detection using an external standard.

The following equipment were used for dissolution tests.

### Dissolution

Dissolution apparatus: Agilent 708-DS with paddles (Apparatus 2, Ph. Eur. 2.9.3.)
Syringe filter: RC Membrane filter, 0.2 μm, 26 mm syringe filters, non-sterile, PP-housing, Luer/slip, Phenomenex
Cannula with filter: 10 Micron Porous (Full Flow) Filters, P/N FIL010-CA-a, QLA
Syringe: 5 ml (6 ml) NORM-JECT, HENKE SASS WOLF

### HPLC

Analytical column: Waters Acquity HSS 1.8 μm, C18 50×2.1 mm
Pump: Waters Acquity ARC Quaternary Solvent Manager (QSM)
Autosampler: Waters Acquity ARC Sample Manager (SM)
Column oven: Waters Acquity ARC Column Manager (CM)
Detector: Waters Acquity ARC PDA Detector
Software: Empower

### Results:

Table 4 shows the dissolution of the tablets as % of nominal content released after 15 minutes. Dissolution was acceptable for all variants with micronized melatonin, but insufficient for tablets with low strength and non-micronized melatonin (table 4, exp. 1D). Since the content was 93.6% in the content uniformity analysis (table 3, exp. 1D), the low dissolution is not caused by low assay, but appears to be due to a slower dissolution due to the larger particle size.

TABLE 4
Table shows the dissolution of the tablets as % of nominal content
released after 15 minutes. Relative standard deviation (RSD) is
given for the released content.

| | Tablet | Melatonin | Average | |
|---|---|---|---|---|
| Experiment | strength | particle size | content | RSD |
| no | (mg/tablet) | (D90, μm) | released (%) | (%) |
| 1A | 1 | 6.0 | 99.25 | 1.24 |
| 1B | 1 | 7.5 | 97.47 | 0.56 |
| 1C | 1 | 9.6 | 98.93 | 0.46 |
| 1D | 1 | 131.8 | 75.37 | 8.52 |
| 1E | 1 | 14.8 | 97.55 | 1.52 |
| 5A | 5 | 6.0 | 100.18 | 0.27 |
| 5B | 5 | 7.5 | 99.68 | 0.60 |
| 5C | 5 | 9.6 | 99.97 | 0.65 |
| 5D | 5 | 131.8 | 99.63 | 0.18 |

### Conclusion:

This example demonstrates that tablets comprising micronized melatonin displayed a satisfactory dissolution, whereas tablets with low strength and non-micronized melatonin had a slow dissolution profile.

From examples 1 and 2 together it can be concluded that formulations containing micronized melatonin display a better uniformity of content and a faster dissolution profile compared to formulations with non-micronized melatonin.

### Example 3: Influence of Lubricant on Tablet Processing and Characteristics

This example investigated how magnesium stearate influences the manufacture process and the properties of the final tablet.

### Method:

### Influence of Magnesium Stearate on Tablet Processing

Tablets of 0.5 mg API (melatonin) were prepared with a composition according to table 5. The ingredients were provided by suppliers according to table 2 of Experiment 1.

TABLE 5

| Strength | 0.5 mg/tablet | |
|---|---|---|
| Ingredient | mg/tablet | % |
| Micronized melatonin | 0.5 | 0.25 |
| Microcrystalline cellulose | 59.6 | 29.8 |
| Mannitol | 135.9 | 67.95 |
| Silica colloidal anhydrous | 1 | 0.5 |
| Magnesium stearate | 3 | 1.5 |
| Total | 200 | 100 |

Formulations with varying amount of magnesium stearate were prepared to enable dry processing of the melatonin formulation to tablets. The formulations were similar to those of table 5 with the exception that magnesium stearate was added and mixed for 2 min at 23 rpm to obtain a magnesium content of 2.25 wt % or 3 wt %. The formulations were prepared as described in example 1 (without addition of disintegrant). The main compression force was 5.2 kN.

### Influence of Magnesium Stearate on Disintegration Time

Tablets of 0.5 mg API (melatonin) were prepared with a composition according to table 6. The ingredients were provided by suppliers according to table 2 of example 1.

TABLE 6

| Strength | 0.5 mg/tablet | |
|---|---|---|
| Ingredient | mg/tablet | % |
| Micronized melatonin | 0.5 | 0.25 |
| Microcrystalline cellulose | 57 | 28.5 |
| Mannitol | 131.5 | 65.8 |
| Silica colloidal anhydrous | 1 | 0.5 |
| Magnesium stearate | 6 | 3 |
| Croscarmellose sodium | 4 | 2 |
| Total | 200 | 100 |

Three experiments were run in parallel (termed F1, F4 and F5, see table 7 below) to test the influence of blending conditions of magnesium stearate on the disintegration time of the tablet.

Ingredients were mixed using a high shear mixer (Diosna 4 litres) according to the following protocol.
1. Mannitol (95%) was sieved through a 1 mm screen into a Diosna vessel 4 liter.
2. Microcrystalline cellulose and melatonin were sieved alternately through a 0.5 mm screen into the mixing container.
3. In the following order, silica colloidal anhydrous, remaining mannitol (5%) and croscarmellose sodium were sieved through a 0.5 mm screen into the mixing container.
4. Mixing step 1: 300 rpm for 2 min
5. Magnesium stearate was sieved through a 1 mm screen into the mixing container.
6. Mixing step 2: mixing speed and time according to table 7

### Results:

### Influence of Magnesium Stearate on Tablet Processing

Tablet compression of the powder mixture comprising 1.5 wt % magnesium stearate caused was interrupted due to die sticking that severely clogged the punch/die sets and hindered production of tablets. Increasing the amount of magnesium stearate to 2.25 wt % improved the processing of tablets but noise due to die sticking was heard after 8 min and tablet compression was interrupted again. Formulations comprising 3 wt % magnesium could be produced with no die sticking in the punch/die sets are no striations or capping of the tablets. Tabletting proceeded for 35 min without interruptions.

### Influence of Magnesium Stearate on Disintegration Time

TABLE 7
Table shows disintegration for tablets produced using different
blending conditions for magnesium stearate.

| | Blending conditions of | | |
|---|---|---|---|
| Experiment | magnesium stearate | | |
| no. | Speed (rpm) | Time (min) | Disintegration time (s) |
| F1 | 500 | 1 | 35 |
| F4 | 300 | 1 | 34 |
| F5 | 500 | 2 | 39 |

For all experiments (F1, F4 and F5) it was observed that the disintegration occurs rapidly and within 40 seconds all tablets have disintegrated. The slightly slower disintegration time of experiment F5 could be due to the magnesium stearate being “over-mixed” in the mixture. The impact of magnesium stearate is that it increases disintegration due to its hydrophobic properties.

### Conclusion:

This example demonstrates that a high amount of magnesium stearate improves the tabletting process. Moreover, the example shows that tablets with fast disintegration time can be produced despite the high amount of magnesium stearate.

### Example 4: Alternative Method of Pre-Mixing Micronized Melatonin with Microcrystalline Cellulose (MCC)

This example investigated an alternative method of premixing of micronized melatonin to improve distribution of melatonin in the solid dosage forms.

### Method:

Tablets of 0.5 mg API (melatonin) were prepared with a composition according to table 6 (as shown in example 3). The ingredients were provided by suppliers according to table 2 of example 1. Ingredients were mixed using two high shear mixers (Diosna 1 and 4 litres) according to the following protocol.

### Pre-Mixing (High Shear Mixer—1 Litre)

1. 30% of the MCC was added to a high shear mixing container of 1 litre.
2. MCC and melatonin were sieved through a 0.5 mm screen into the mixing container in the following order:
   MCC (equal volume as melatonin)
   Melatonin
   MCC (equal volume as melatonin)
3. 30% of the remaining MCC (30%) was added to the mixing container.
4. Mixing: 300 rpm for 2 min

### Mixing (High Shear Mixer—4 Litre)

5. Mannitol (50%) was sieved through a 1 mm screen into a high shear mixing container of 4 litre
6. The components below were sieved through a 0.5 mm screen into the mixing container in the following order:
   Half of the remaining MCC
   Pre-mix
   Croscarmellose sodium
   Silica colloidal anhydrous
   The remaining amount of MCC
7. The remaining amount of mannitol (50%) was sieved through a 1 mm screen into the mixing container
8. Mixing: 300 rpm for 2 min
9. Magnesium stearate was sieved through a 1.0 mm sieve into the mixing container
10. Mixing: 500 RPM for 1 min.

The resulting powder blend was compressed to tablets as described in example 1. Analysis of content uniformity was performed as described in example 1.

### Results:

Tablets manufactured by the alternative pre-mixing method described in this example yielded a content uniformity of 99.57% with a relative standard deviation of 0.6%. This gave an acceptance value (AV) according to the European Pharmacopoeia 10^{th} Edition of 1.5.

### Conclusion:

This example demonstrates that more than a single method comprising stepwise addition of micronized melatonin and MCC can be used to provide melatonin formulations with high content uniformity.

### Example 5: Influence of Compression Force on Bioavailability of the Melatonin Formulation

This example investigated the influence of the compression force on the disintegration time and dissolution profile of the melatonin formulation.

### Method:

Melatonin Formulations without a Disintegrant

Three formulations (N4, N7 and N10, see table 8) at different melatonin dose were prepared to test the effect of compression force on disintegration time of the formulation.

TABLE 8

| | N4 | | N7 | | N10 | |
|---|---|---|---|---|---|---|
| Ingredient | mg/tablet | % | mg/tablet | % | mg/tablet | % |
| Micronized melatonin | 5.0 | 2.5 | 0.5 | 0.25 | 3.0 | 1.5 |
| Microcrystalline cellulose | 57.24 | 28.62 | 58.12 | 29.06 | 57.92 | 28.96 |
| Mannitol | 130.56 | 65.28 | 132.58 | 66.29 | 132.08 | 66.04 |
| Silica colloidal anhydrous | 0.2 | 0.1 | 1.8 | 0.9 | 1.0 | 0.5 |
| Magnesium stearate | 7.0 | 3.5 | 7.0 | 3.5 | 6.0 | 3.0 |
| Total | 200 | 100 | 200 | 100 | 200 | 100 |

The three formulations were prepared according to the mixing procedure below, involving formation of a pre-mixture comprising micronized melatonin and MCC, and subsequent mixing with the remaining ingredients.

### Pre-Mixing (High Shear Mixer—1 Litre)

1. Melatonin and an equal volume of MCC was mixed with a spatula in a beaker and transferred to the 1 L Diosna mixing container with about half the amount of total MCC.
2. Mixing at 300 rpm was made for 2 min and the inside of the mixing container and impeller were scraped with a scrape card.
3. Approx. 100 g MCC was added and mixed for 3 min, after which the inside of the mixing container and impeller were scraped with a scrape card.
4. The rest of MCC was added and mixed for 3 min after which scraping was performed. This step was repeated one more time for an additional mixing of 3 min.

### Powder Mixing (Tumbling Mixer—5 Litres)

1. The pre-mixture was sieved through a 500 μm screen and transferred to the 5 L mixing container of the Turbula mixer between a bottom and a top layer of mannitol (about equal volumes of mannitol in the two layers). Mannitol was delumped by sieving through a 1.00 mm screen.
2. Mixing was made at 32 rpm for 5 min.
3. Glidant was roughly mixed with a similar volume of the powder mixture in a plastic bag and sieved through 500 μm.
4. The glidant mixture was added to the 5 L mixing container and blending for 3 min at 32 rpm was performed.
5. Magnesium stearate was pre-mixed with a similar volume of the mixture above, sieved through 500 μm and added to the 5 L mixing container. Blending for 2 min at 32 rpm was performed.

### Tablet Compression

Tablets were compressed in 8 mm circular punch/die sets (4 sets, normal cup depth). Filling corresponding to a target tablet weight of 200 mg was employed. Main compression force approx. 5 kN, turret speed: 20 rpm and filler speed: 22 rpm were applied. Tablets were also made at a lower compression force—approx. 4 kN (3.8-4.9 kN)—and a higher force—about 6 kN (5.5-6.6 kN), and a highest force of >8 kN.

### Dissolution Testing

Determination of dissolution of tablets were performed as described in example 2.

### Melatonin Formulations Including a Disintegrant

Two melatonin formulations (exp. no. 1 and 3, see table 9) with different amount of disintegrant (1.5 wt % and 3.0 wt %) were prepared to test the influence of the disintegrant on disintegration time and dissolution of the formulation.

TABLE 9

| Experiment no | 1 | | 3 | |
|---|---|---|---|---|
| Ingredient | mg/tablet | % | mg/tablet | % |
| Micronized melatonin | 3 | 1.5 | 5 | 2.5 |
| Microcrystalline cellulose | 57.0 | 28.5 | 55.5 | 27.8 |
| Mannitol | 130.0 | 65.0 | 126.5 | 63.2 |
| Silica colloidal anhydrous | 1 | 0.5 | 1 | 0.5 |
| Magnesium stearate | 6 | 3 | 6 | 3 |
| Croscarmellose sodium | 3 | 1.5 | 6 | 3 |
| Total | 200 | 100 | 200 | 100 |

The two formulations were prepared according to the mixing procedure described above for the three formulations without disintegrant (N4, N7, N10) with the sole exception that croscarmellose sodium was mixed with a similar volume of mannitol in a plastic bag and sieved through a 500 μm screen as part of step 1 in the powder mixing part of the procedure.

Tablet compression and dissolution testing was performed as for the three formulations without disintegrant (N4, N7, N10) above.

### Results:

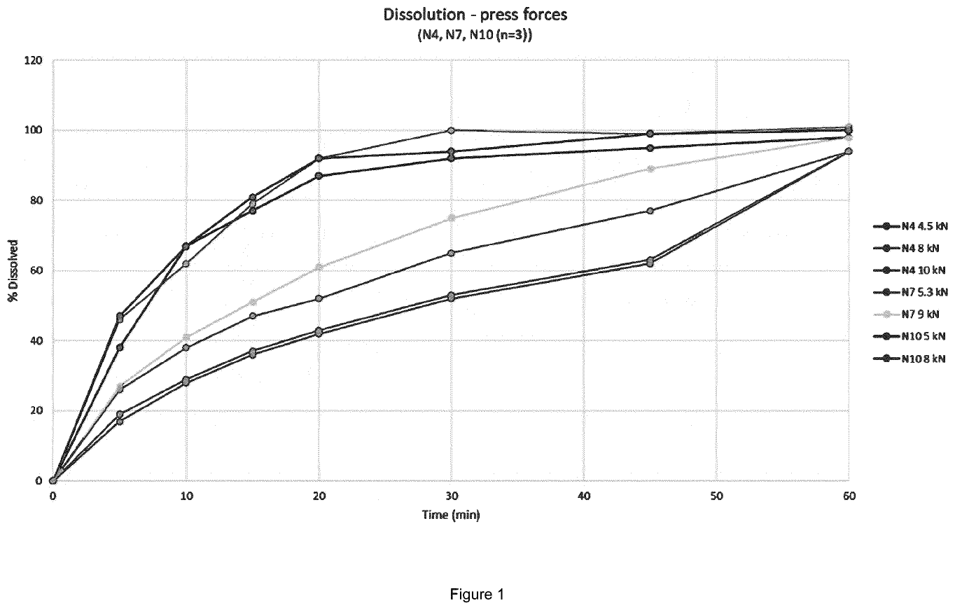

The dissolution profiles obtained for the three melatonin formulations without a disintegrant (N4, N7, and N10) showed and strong dependency on compression force. FIG. 1 show a subset of the data and demonstrate that formulations compressed with compression force ≥8 kN had significantly delayed dissolution profiles. Only one of the three formulations (N7, low level of API) compressed at >8 kN fulfilled the limit of quality target product profile (QTPP) Q>75% at 45 min (i.e. >80% dissolved).

Addition of croscarmellose sodium (Cam) to the melatonin formulation resulted in fast disintegration times, even as the compression force was increased (see table 10). The disintegration time increased when the compression force was increased, which is expected, but disintegration time was kept below 50 seconds.

TABLE 10
Table shows disintegration time for formulations of different amount
of disintegrant and compressed at increasing compression force.

| | | Disintegration time (s) | | |
|---|---|---|---|---|
| Experiment | | Compression | Compression | Compression |
| no. | Formulation | force = 5 kN | force = 6.5 kN | force = 8 kN |
| 1 | 3 mg, | 28 | 39 | 45 |
| | 1.5% Cam | | | |
| 3 | 5 mg, | 26 | 37 | 48 |
| | 3% Cam | | | |

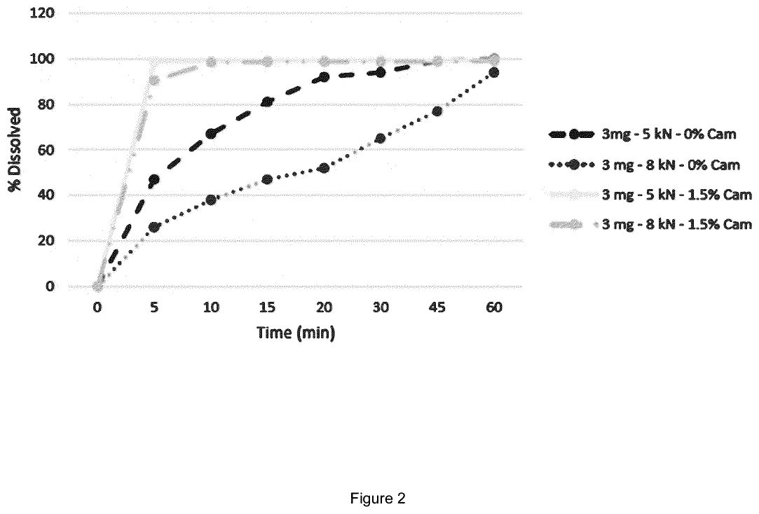

The dissolution profile of experiment no. 1 as compared to N10 (without disintegrant) reflected the fast disintegration time and showed a significant improvement in dissolution by addition of a disintegrant (FIG. 2). Notably, rapid dissolution was achieved for the formulations with a disintegrant also for tablets compressed at high compression force.

### Conclusion:

This example demonstrates that disintegration time can be reduced, and the dissolution profile improved by addition of a disintegrant to the melatonin formulation. Moreover, inclusion of a disintegrant greatly reduces the sensitivity to compression force compared to formulations without a disintegrant.

### Example 6: Characteristics of Rapid Release Melatonin Formulation

This example investigated the properties of the tablets obtained by the present method and evaluated the suitability of the tablets as a rapid release melatonin formulation.

### Method:

The melatonin formulations were prepared as described in example 1. Herein are presented only the formulations with micronized melatonin that allow uniform distribution of content across all tablet strengths.

The compression force was varied to investigate the influence on disintegration time and physical properties of the tablets.

It is noted that for large scale production a Fette 2090 tablet press would be used instead of the Fette 52i tablet press.

### Results:

Results are summarised in table 11.

No major differences in compression force were seen between the experiments, all were centred around approximately 4 kN, 6 kN and 7.5 kN.

Resistance to crushing increases as the compression force increases, which is expected, and all the numbers indicate good manufacturability for all formulations.

Friability decreases as the compression force increases as expected. Friability is low for all formulations and well within acceptable tablet values.

The disintegration results show that higher compression forces result in longer disintegration times. All formulations have fast disintegration times of less than 40 seconds, even at the highest compression force. Thus, the formulations are well suited for rapid release of melatonin.

TABLE 11
Table shows properties of rapid release melatonin formulations.

| Experiment | Compression | Resistance to | Friability | Disintegration |
|---|---|---|---|---|
| no. | force (kN) | crushing (N) | (%) | time (s) |
| 1A | 4.24 | 42 | 0.49 | 18 |
| | 6.20 | 61 | 0.31 | 29 |
| | 7.68 | 76 | 0.32 | 34 |
| 1B | 4.18 | 41 | 0.39 | 17 |
| | 5.91 | 59 | 0.33 | 24 |
| | 7.22 | 75 | 0.34 | 31 |
| 1C | 4.17 | 40 | 0.41 | 16 |
| | 5.69 | 58 | 0.37 | 21 |
| | 7.66 | 83 | 0.30 | 33 |
| 1E | 3.89 | 40 | 0.50 | 18 |
| | 5.81 | 58 | 0.38 | 23 |
| | 8.16 | 84 | 0.30 | 34 |
| 5A | 4.25 | 41 | 0.45 | 16 |
| | 6.18 | 64 | 0.35 | 24 |
| | 7.41 | 79 | 0.25 | 27 |
| 5B | 4.29 | 45 | 0.40 | 14 |
| | 6.24 | 65 | 0.34 | 23 |
| | 7.18 | 76 | 0.29 | 27 |
| 5C | 4.25 | 43 | 0.35 | 16 |
| | 6.18 | 63 | 0.28 | 25 |
| | 7.49 | 81 | 0.31 | 29 |

### Conclusion:

This example demonstrates that high quality rapid release melatonin formulations can be prepared using the method described herein. The rapid release melatonin formulations have fast disintegration time that promote quick bioavailability of melatonin.

1. A method for manufacture of a rapid release melatonin formulation in solid dosage form, said method comprising:
i) a first adding step comprising adding first components into a mixing container to form a first mixture, wherein the first components comprise one or more fillers and optionally one or more glidants and/or disintegrants,
ii) a stepwise adding step comprising providing a first amount of microcrystalline cellulose and a total amount of micronized melatonin, and adding said first amount of microcrystalline cellulose and said total amount of micronized melatonin into the mixing container stepwise in multiple portions,
iii) a second adding step comprising adding second components into the mixing container to form a second mixture, wherein the second components comprise a second amount of microcrystalline cellulose and optionally one or more fillers, glidants and/or disintegrants, and wherein the second mixture comprises micronized melatonin, microcrystalline cellulose, filler, glidant and disintegrant,
iv) a third adding step comprising adding magnesium stearate into the mixing container to form a final mixture, and
v) a processing step comprising processing the final mixture to a solid dosage form,
wherein at least one of the first and/or second adding step comprises adding one or more glidants, and
wherein at least one of the first and/or second adding step comprises adding one or more disintegrants.

2. The method according to claim 1, wherein the second adding step is followed by a first blending of the second mixture for a duration of 40-240 seconds.

3. The method according to claim 1, wherein the ratio between said first amount of microcrystalline cellulose and said second amount of microcrystalline cellulose is in the range of 1:4 to 1:10 wt %/wt %, preferably approximately 1:5 wt %/wt %, with respect to the total weight of microcrystalline cellulose in the solid dosage form.

4. The method according to claim 1, wherein each portion of the stepwise adding step comprises a fraction of said first amount of microcrystalline cellulose and a fraction of said total amount of micronized melatonin.

5. The method according to claim 4, wherein said fraction of said first amount of microcrystalline cellulose and said fraction of said total amount of micronized melatonin are sieved together into the mixing container through a pre-mixing screen.

6. The method according to claim 1, wherein said multiple portions are three portions, four portions, five portions, six portions, seven portions, or eight portions, preferably five portions.

7. The method according to claim 1, said method comprising the steps of:
i) a first adding step comprising adding first components comprising a filler into a mixing container to form a first mixture,
ii) a stepwise adding step comprising adding providing a first amount of microcrystalline cellulose and a total amount of micronized melatonin, and adding said first amount of microcrystalline cellulose and said total amount of micronized melatonin into the mixing container stepwise in multiple portions,
iii) a second adding step comprising adding second components comprising a second amount of microcrystalline cellulose, a glidant and a disintegrant into the mixing container to form a second mixture,
iv) a third adding step comprising adding magnesium stearate into the mixing container to form a final mixture, and
v) a processing step comprising processing the final mixture to a solid dosage form.

8. The method according to claim 1, wherein the final mixture comprises at least 3 wt % magnesium stearate with respect to the total weight of the solid dosage form.

9. The method according to claim 1, wherein the third adding step is followed by a second blending of the final mixture for less than 120 seconds.

10. The method according to claim 1, wherein the filler is mannitol, the glidant is colloidal anhydrous silica, and the disintegrant is croscarmellose sodium.

11. A rapid release melatonin formulation obtainable by a method according to claim 1.

12. A rapid release melatonin formulation comprising:
0.2-3 wt % micronized melatonin,
20-35 wt % microcrystalline cellulose,
65-75 wt % of one or more fillers,
0.2-0.8 wt % of one or more glidants,
1-3 wt % of one or more disintegrants, and
at least 3 wt % magnesium stearate,
wherein the formulation is provided as solid dosage form and wt % is with respect to the total weight of the formulation.

13. The rapid release melatonin formulation according to claim 12, wherein the filler is mannitol, the glidant is colloidal anhydrous silica, and the disintegrant is croscarmellose sodium.

14. The rapid release melatonin formulation according to claim 12, wherein the solid dosage form is selected from the group consisting of a tablet, pill, lozenge, capsule, and pastille, preferably a tablet.

15. (canceled)

16. (canceled)

17. A kit of parts comprising:
a plurality of solid dosage forms comprising a rapid release melatonin formulation according to claim 11, and
optionally, instructions for use.

18. A method of treating a sleep disorder in a subject, the method comprising administering to the subject the rapid release melatonin formulation of claim 11.

19. A method of treating a disorder associated with disruption of circadian rhythm in a subject, the method comprising administering to the subject the rapid release melatonin formulation of claim 11.

20. A method of treating a psychiatric disorder in a subject, the method comprising administering to the subject the rapid release melatonin formulation of claim 11.
